(19) United States
(12) Patent Application Publication
(10) Pub. No.: US 2018/0037605 A1
(43) Pub. Date: Feb. 8, 2018

(54) SYNTHETIC PEPTIDES AND ENZYMATIC FORMATION OF INTRACELLULAR HYDROGELS

(71) Applicant: BRANDEIS UNIVERSITY, Waltham, MA (US)

(72) Inventors: Xuewen DU, Waltham, MA (US); Jie ZHOU, Waltham, MA (US); Bing XU, Newton, MA (US)

(21) Appl. No.: 15/550,649

(22) PCT Filed: Feb. 26, 2016
(86) PCT No.: PCT/US16/19866

§ 371 Date: Aug. 11, 2017

Related U.S. Application Data
(60) Provisional application No. 62/121,237, filed on Feb. 26, 2015.

(51) Int. Cl. C07K 5/02; A61K 47/64; C12P 21/06

(52) U.S. Cl. CPC C07K 5/0202; C12P 21/06; A61K 47/64; A61K 38/00

(57) ABSTRACT

The invention relates to a peptide that includes a plurality of amino acid residues and an enzymatically cleavable moiety including taurine or hypotaurine, the enzymatically cleavable-moiety being linked to the peptide via covalent bond, wherein the peptide is capable of self-assembly to form nanofibrils in the presence of an enzyme that hydrolyzes the enzymatically cleavable-moiety. Compositions containing the enzymatically responsive peptide, and the use thereof for forming a nanofibril network internally of cells, for treating a cancerous condition, and imaging cells are also disclosed.

This application claims the benefit of U.S. Provisional Patent Application Ser. No. 62/121,237, filed Feb. 26, 2015, which is hereby incorporated by reference in its entirety.

The present invention was made with support from the National Institutes of Health under grant R01CA142746. The U.S. government has certain rights in this invention.

### BACKGROUND OF THE INVENTION

Few pathways are readily available for the biological utilization of D-amino acids. Amino acids of importance rarely exist in nutrition as D-isomers, and naturally occurring proteins consist exclusively of L-amino acids. Although microorganisms, marine invertebrates, and a few other animals do synthesize D-amino acids and, during food processing, racemization occurs to produce D-amino acids, these examples remain as exceptions (Friedman et al., Amino Acids 42:1553-1582 (2012)). This unique feature of D-amino acids endows D-peptides with enduring biostability due to their resistance against endogenous proteases in vitro and in vivo. D-peptides, thus, are gaining increasing attention and readily find applications in a variety of areas of biology and biomedicine (Reich et al., J. Am. Chem. Soc. 118:6345-6349 (1996); Morii et al., J. Am. Chem. Soc. 124:180-181 (2002); Michaud et al., J. Am. Chem. Soc. 125:8672-8679 (2003); Eckert et al., Cell 99:103-115 (1999); Schumacher et al., Science 271:1854-1857 (1996); Fitzgerald et al., J. Am. Chem. Soc. 117:11075-11080 (1995)). For example, D-peptides have found applications for tracing the lineage of cells (Weisblat et al., Science 209:1538-1541 (1980)) and the growth of axons (Mason et al., Nature 296:655-657 (1982)), disrupting protein interactions (Liu et al., Proc. Natl. Acad. Sci. U.S.A 107:14321-14326 (2010); McDonnell et al., Nat. Struct. Biol. 3:419-426 (1996); Merrifield et al., Proc. Natl. Acad. Sci. U.S.A 92:3449-3453 (1995)), reducing adverse drug reactions of anti-inflammatory drugs (Li et al., J. Am. Chem. Soc. 135:542-545 (2013)), and serving as a medium for drug delivery (Liang et al., Langmuir 25:8419-8422 (2009)).

These above advances of the use of D-amino acid-based materials mainly occur for extracellular applications, because cellular uptake of D-peptides is ineffective (Ishida et al., J. Am. Chem. Soc. 135:12684-12689 (2013); Hayashi et al., J. Am. Chem. Soc. 135:12252-12258 (2013)). A recent report also reveals that D-peptides exclusively enrich in cell membrane (Wang et al., ACS Appl. Mater. Interfaces 6(12):9815-9821 (2014)). Although it is feasible to inject D-peptides directly into cells (Weisblat et al., Science 209:1538-1541 (1980); Mason et al., Nature 296:655-657 (1982)), microinjection is unsuitable for common mammalian cells, and is impractical to be applied to large numbers of cells including when administering to an animal. To further explore the merits of D-peptides inside cells, it is essential to develop an effective strategy to enhance the cellular uptake of D-peptides. Although there are reports of cell penetrating peptides (CPPs), e.g., poly(lysine), poly(arginine) or poly(D-arginine) (Mitchell et al., J. Pept. Res. 56:318-325 (2000); Wender et al., Adv. Drug Deliver Rev. 60:452-472 (2008); Rothbard et al., J. Am. Chem. Soc. 126:9506-9507 (2004)), these CPPs still have limitations, such as their susceptibility to metabolic degradation, dependence on cell lines and cellular differentiation state, or poor cellular compatibility (especially of those cationic CPPs) (Richard et al., J. Biol. Chem. 278:585-590 (2003); Sakai et al., J. Am. Chem. Soc. 125:14348-14356 (2003)). Thus, there remains an unmet need of new molecular promoters for enhancing cellular uptake of D-peptides, as well as other bioactive molecules.

The present application overcomes these and other deficiencies in the art.

### SUMMARY OF THE INVENTION

A first aspect of the invention relates to a peptide that includes a plurality of amino acid residues and an enzymatically cleavable moiety including a taurine or hypotaurine residue, the enzymatically cleavable-moiety being linked to the peptide via covalent bond, wherein the peptide is capable of self-assembly to form nanofibrils in the presence of an enzyme that hydrolyzes the enzymatically cleavable-moiety.

In certain embodiments, the peptide includes a plurality of aromatic amino acids.

In certain embodiments, the peptide includes a fluorophore conjugated to the peptide.

In certain other embodiments, the peptide includes a cytotoxic agent (including chemotherapeutic agents, antiangiogenic agents, and immunomodulating agents) conjugated to the peptide.

A second aspect of the invention relates to a pharmaceutical composition including a pharmaceutically acceptable carrier and a peptide according to the first aspect of the invention. One or more structurally distinct peptides can be included.

In certain embodiments, the pharmaceutical composition may also include an effective amount of a cytotoxic agent (including chemotherapeutic agents, antiangiogenic agents, and immunomodulating agents). Here, the cytotoxic agent is not conjugated to the peptide.

A third aspect of the invention relates to a method for treating a cancerous condition. This method includes administering to a subject having a cancerous condition a therapeutically effective amount of a peptide according to the first aspect or a pharmaceutical composition according to the second aspect, wherein said administering is effective to cause uptake of the peptide by the cancer cells and intracellular self-assembly of the peptides to form a nanofibril network upon enzymatic cleavage of the enzymatically cleavable-moiety.

A fourth aspect of the invention relates to a method for forming a nanofibril network internally of cells. This method includes contacting a cell that expresses an endoenzyme having esterase/hydrolase activity with a peptide according to the first aspect or a pharmaceutical composition according to the second aspect, wherein the contacting is effective to cause self-assembly of the peptides to form an intracellular nanofibril network within the contacted cell.

In certain embodiments, the cell is a cancer cell, in which case the contacting step is effective to inhibit cancer cell migration, inhibit cancer cell survival, and/or inhibit cancer cell growth.

A fifth aspect of the invention relates to a method for cellular imaging that includes contacting a cell that expresses an endoenzyme having esterase/hydrolase activity with a peptide according to the first aspect, where the peptide includes a fluorophore conjugated to the peptide, wherein said contacting is effective to cause self-assembly of the peptides to form an intracellular nanofibril network within the cell; and obtaining an image of the contacted cells, which exhibit concentration-dependent fluorescence by fluorophores within the intracellular nanofibril network.

A sixth aspect of the invention relates to a method of making a peptide of the present invention, which includes: providing a peptide comprising a plurality of amino acid residues covalently linked to an enzymatically cleavable moiety having a terminal reactive group, and reacting the peptide with taurine or hypotaurine to form a peptide according to the first aspect of the invention.

The accompanying Examples demonstrate that taurine, a natural and non-proteinogenic amino acid, drastically boosts the cellular uptake of D-peptides in mammalian cells by more than 10 fold, from μM (without the conjugation of taurine to D-peptide) to over mM (after the conjugation of taurine to the D-peptide). The uptake of a large amount of the ester conjugate of taurine and D-peptide allows intracellular esterase to trigger intracellular self-assembly of the D-peptide derivative, which further enhances the cellular accumulation of the D-peptide derivative. Differing fundamentally from the usually positive charged cell-penetrating peptides, the biocompatibility, stability, and simplicity of enzyme cleavable taurine (or hypotaurine) motif promises a new way to promote the uptake of bioactive molecules for countering the action of efflux pump and contributing to intracellular molecular delivery.

The co-incubation of one of the inventive peptides, designated D-10, at an effective concentration with cisplatin significantly boosted the activity of cisplatin against SKOV3 and A2780cis, two lines of drug resistant ovarian cancer cells. The efficacy of this approach (inhibiting over 80% of SKOV3 by 20 μM of cisplatin and 15 μg/mL of D-10) is, in fact, comparable to that of the innovative approach based on the co-delivery of siRNA and cisplatin nanoparticles (80% inhibition of SKOV3 by 75 μM of cisplatin) (He et al., J. Am. Chem. Soc. 136:5181-5184 (2014), which is hereby incorporated by reference in its entirety). The Examples also extend the utility of D-10 with other cancer cell lines that express intracellular carboxylesterase activity, either alone or in combination with cisplatin. Thus, the present invention affords a synergistic combination therapy for numerous forms of cancer that exhibit intracellular esterase activity.

### BRIEF DESCRIPTION OF THE INVENTION

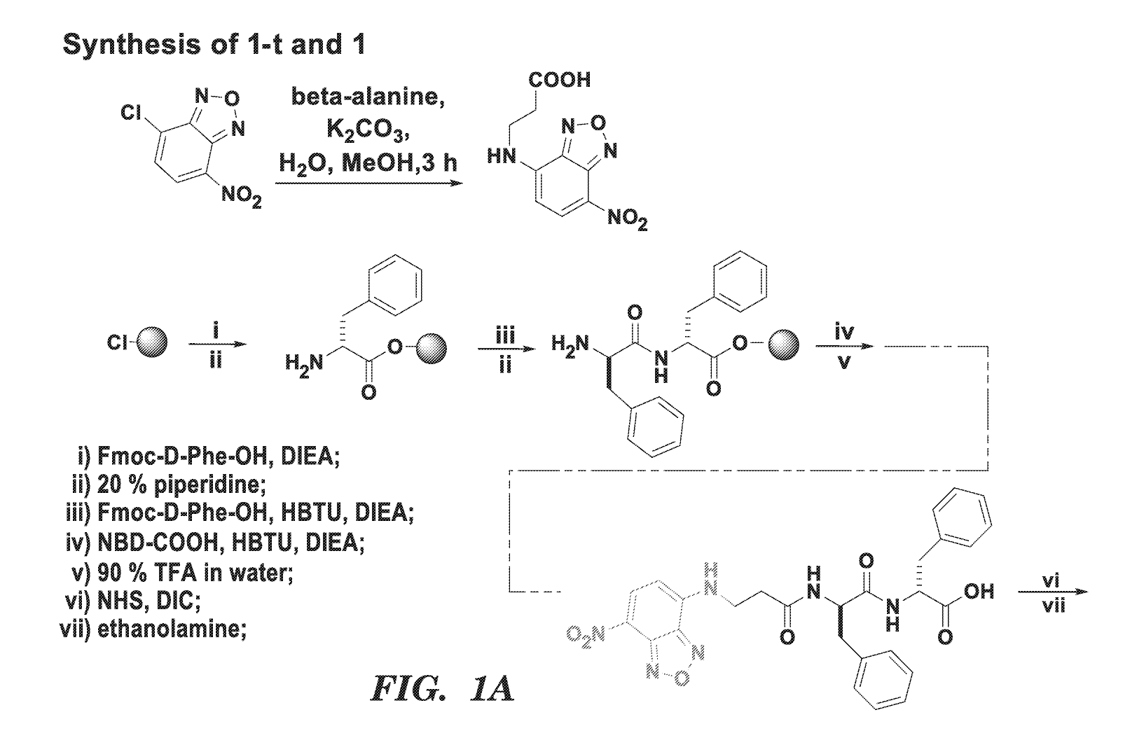

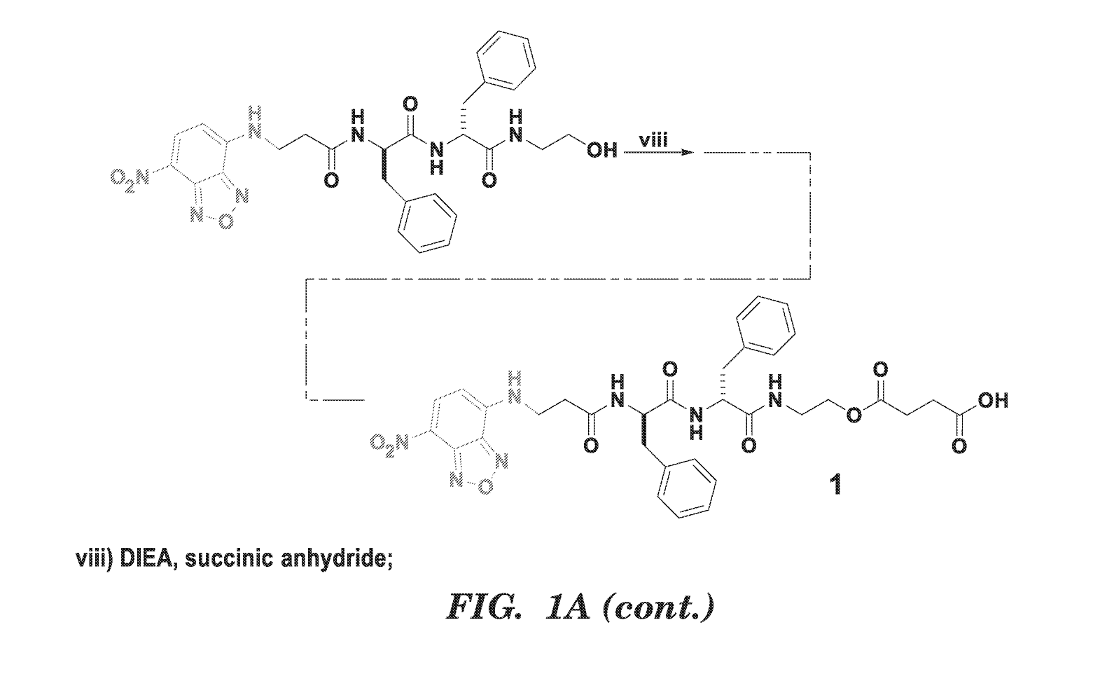

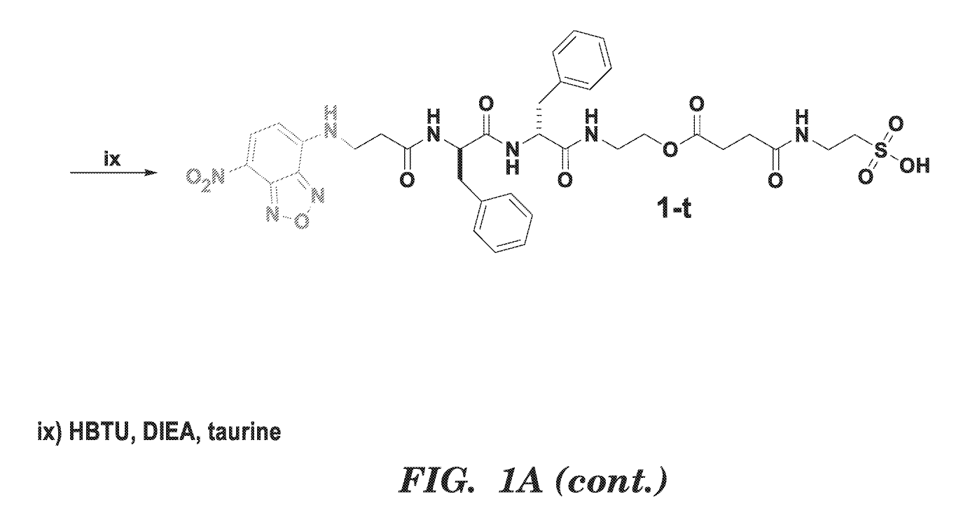

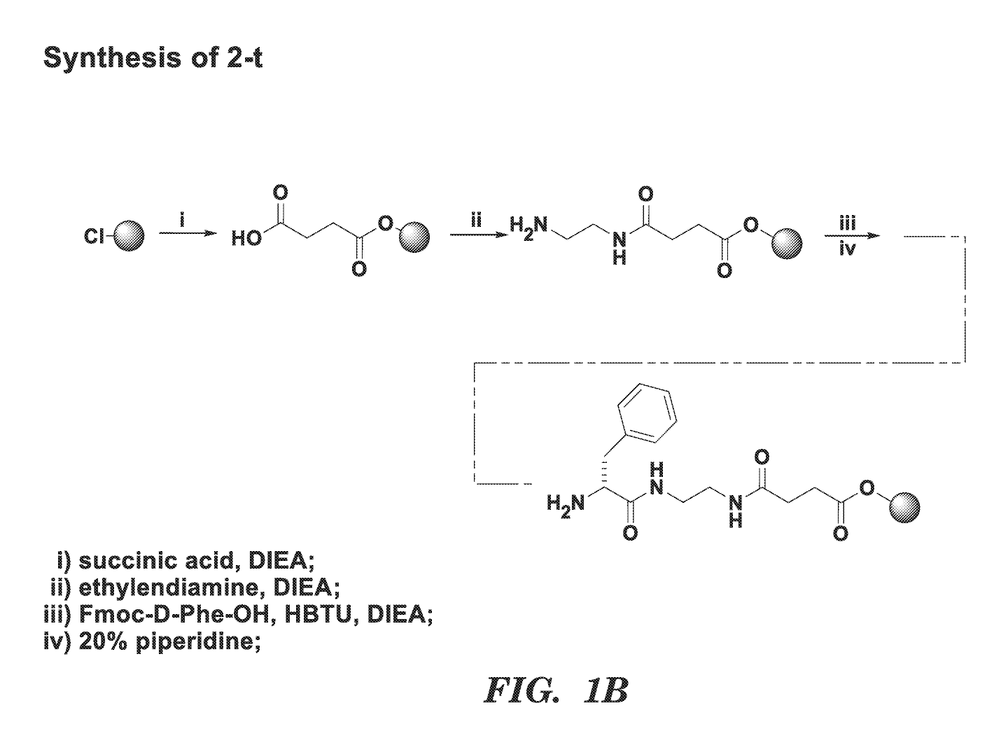

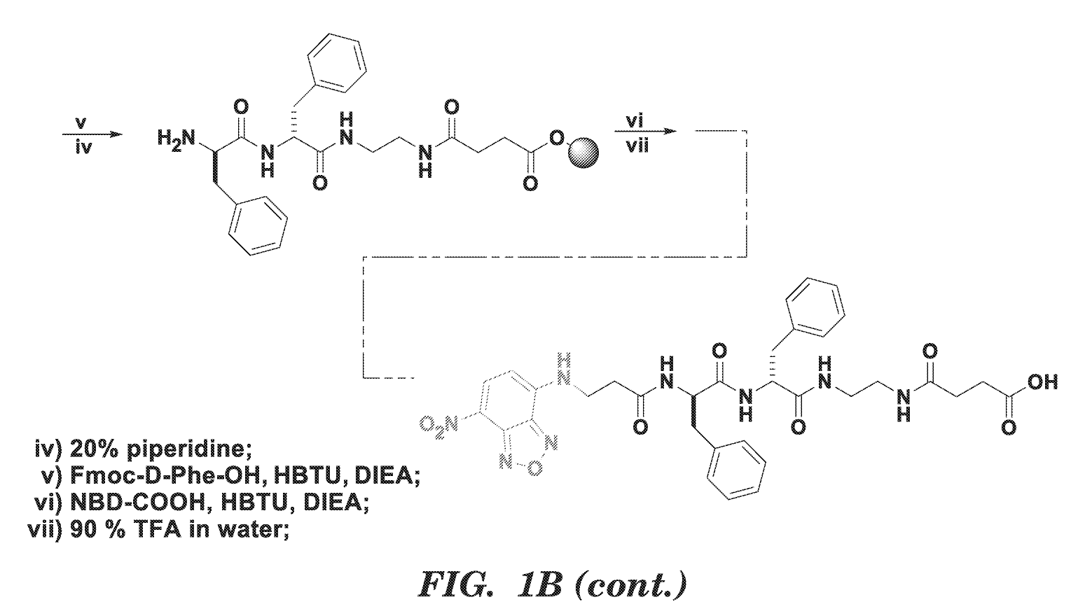

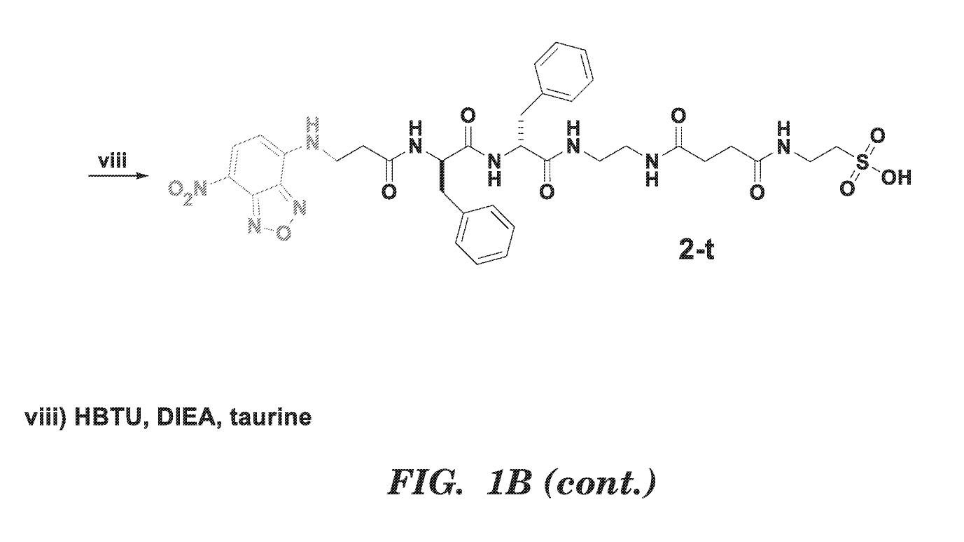

FIG. 1A-B illustrate the synthetic routes for the following compounds: 3-((7-nitrobenzo(c)-1,2,5-oxadiazol-4-yl)amino) (“NBD”)-proprionyl-F_{D}F_{D} bearing a C-terminal ester-linked taurine conjugate (compound designated as 1-t), NBD-proprionyl-F_{D}F_{D} bearing a C-terminal ester-linked proprionic acid group (compound designated as 1), and NBD-proprionyl-F_{D}F_{D} bearing a C-terminal amide-linked taurine conjugate (compound designated as 2-t).

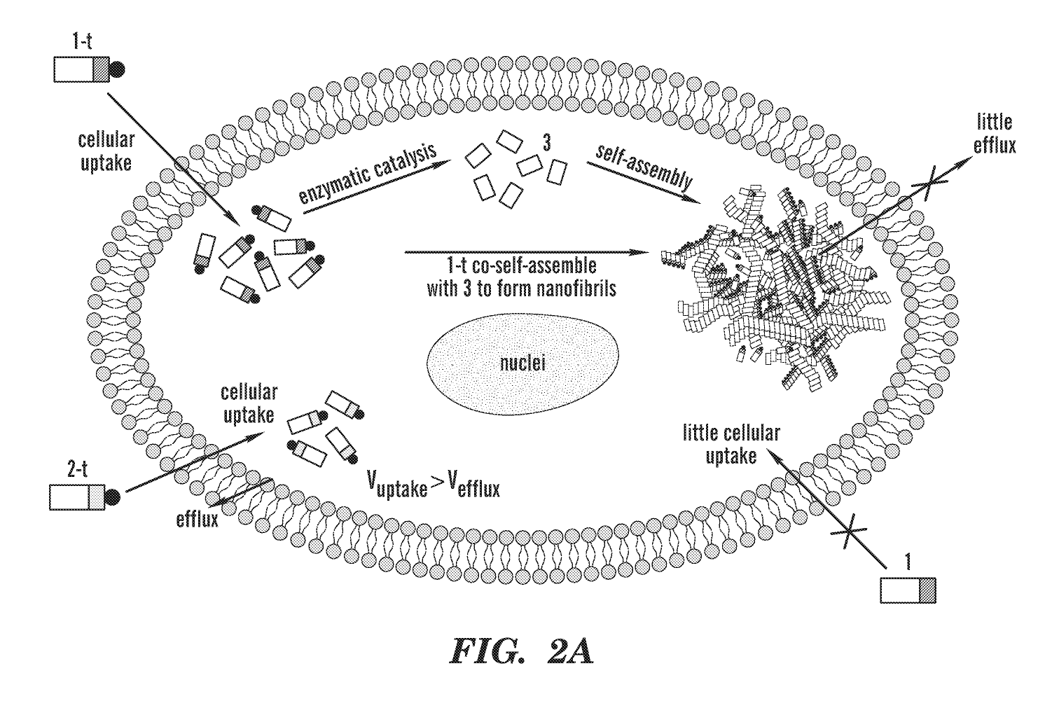

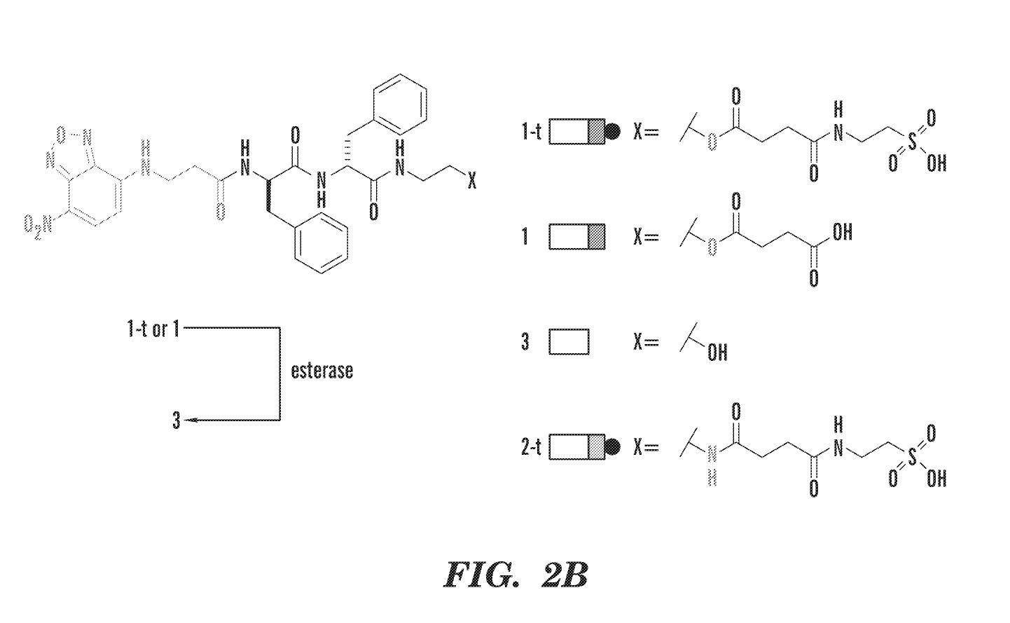

FIGS. 2A-B illustrate the mechanism of action for precursor (1-t), the corresponding hydrogelator (3), and the relevant control molecules (1 & 2-t). FIG. 2A schematically illustrates the ability of taurine conjugation to boost cellular uptake of a D-peptide precursor and the subsequent enzyme-catalyzed self-assembly to form supramolecular nanofibers, accumulating inside cells. FIG. 2B shows the molecular structures of the precursor (1-t), the corresponding hydrogelators (3), and the relevant control molecules (1 & 2-t).

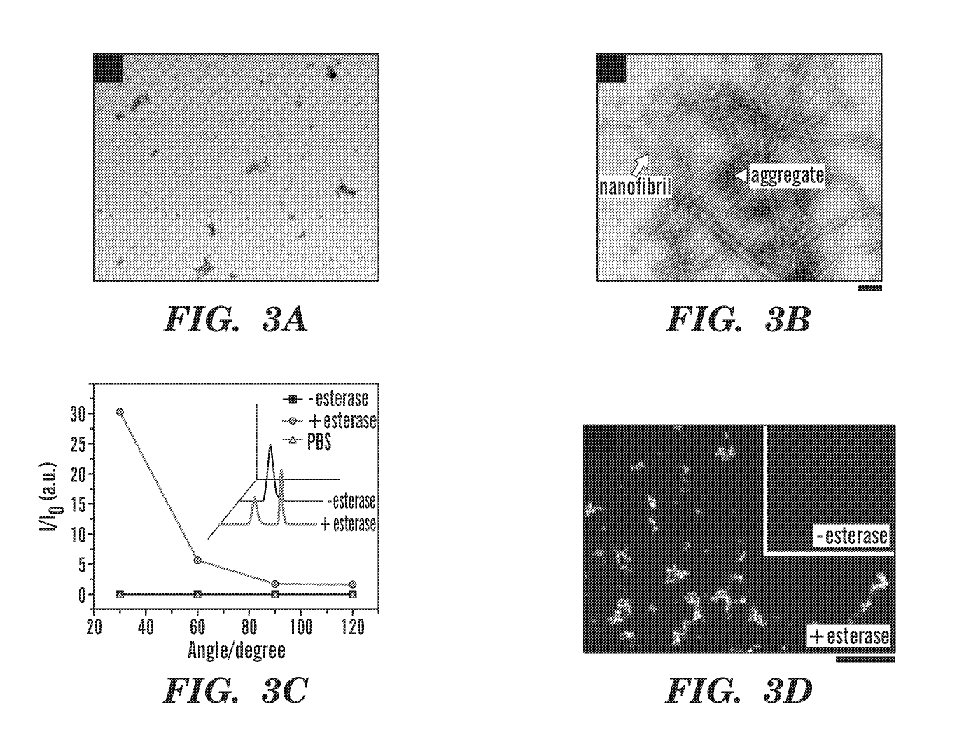

FIG. 3A-D illustrate the in vitro effects of enzymatic cleavage of 1-t. TEM images of the solutions of 1-t (100 μM) in PBS buffer before (FIG. 3A) and after (FIG. 3B) the addition of esterase. The scale bar is 100 nm. FIG. 3C shows the static light scattering (SLS) signals of the solution of 1-t (100 μM) in PBS buffer without and with the addition of esterase. Inset is the corresponding analytical HPLC traces of the solutions. FIG. 3D is a confocal fluorescent microscope image (×20 dry objective lens), which shows the appearance of bright spots in the solution of 1-t (100 μM, pH 7.4) after the addition of esterase. Inset is the corresponding image of the solution before the addition of esterase. In FIGS. 3A-D, the addition of esterase is for 24 hours and at 1 U/mL. The scale bar is 100 μm.

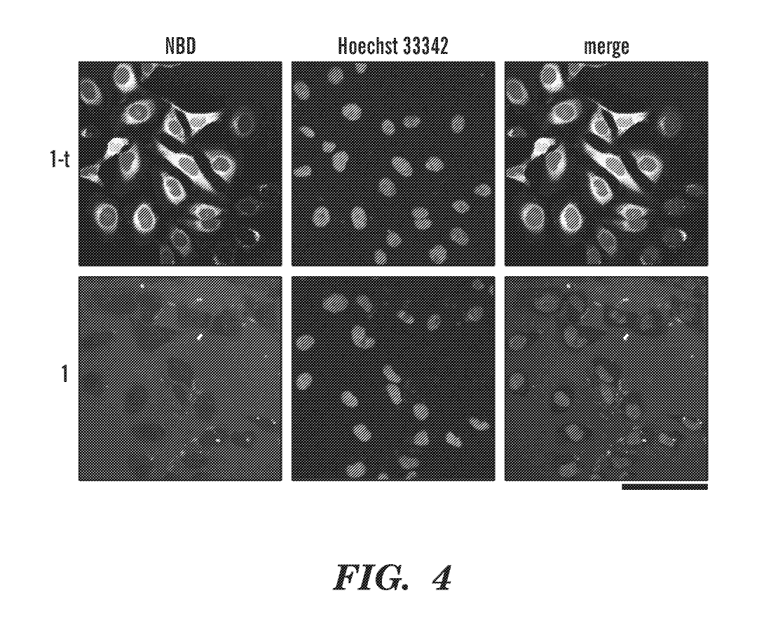

FIG. 4 is a panel of fluorescent confocal microscopy images illustrating the fluorescence emission in HeLa cells with the treatment of 1-t (upper) and 1 (bottom) at the concentration of 200 μM in culture medium for 24 hours and co-stained with Hoechst 33342 (nuclei).

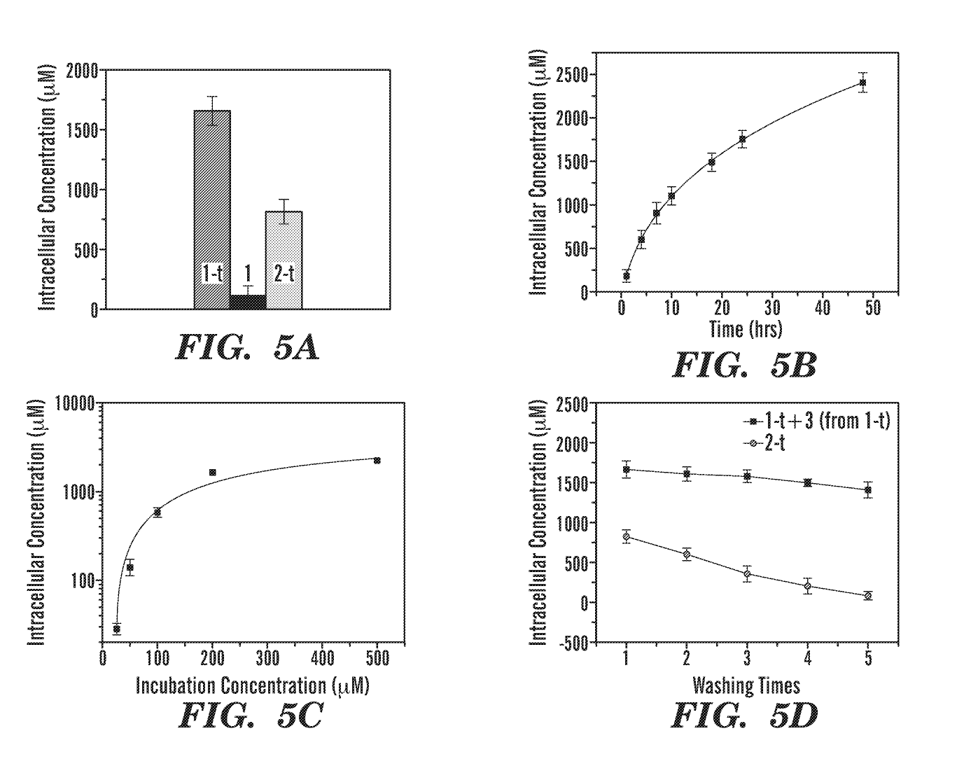

FIGS. 5A-D are graphs illustrating cell uptake of 1-t, 1, and 2-t by HeLa cells. FIG. 5A is a bar graph showing the uptake concentration of 1-t, 1, and 2-t inside the HeLa cells after incubation with corresponding compounds at the concentration of 200 μM in culture medium for 24 hours. The Cᵤ of 1-t or 1 is the sum of 3 and 1-t (or 1) inside the cells. FIG. 5B is a graph of cellular uptake of 1-t (200 μM) at different time points. FIG. 5C is a graph of cellular uptake of 1-t at different incubation concentration at the time point of 24 hours. FIG. 5D is a graph of Cᵤ of 1-t and the concentration 2-t after washing the cells with PBS buffer at each wash.

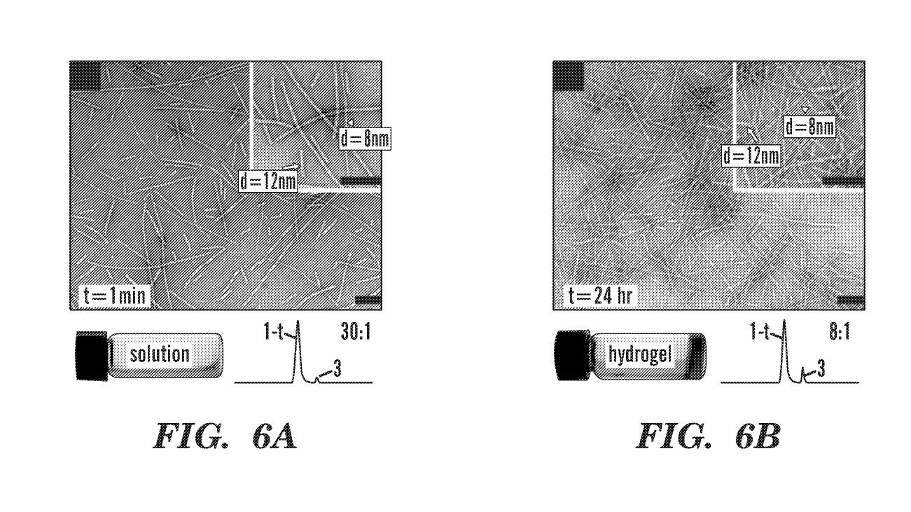

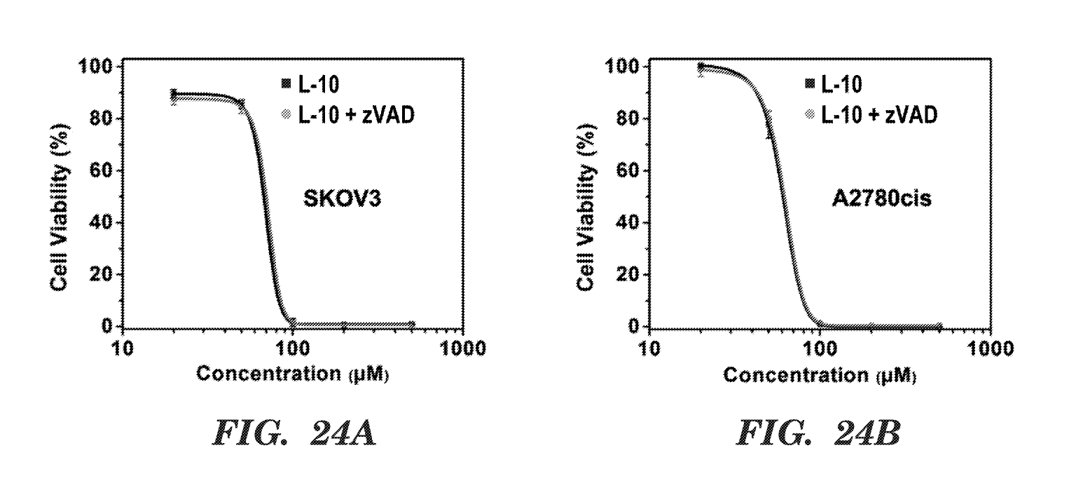

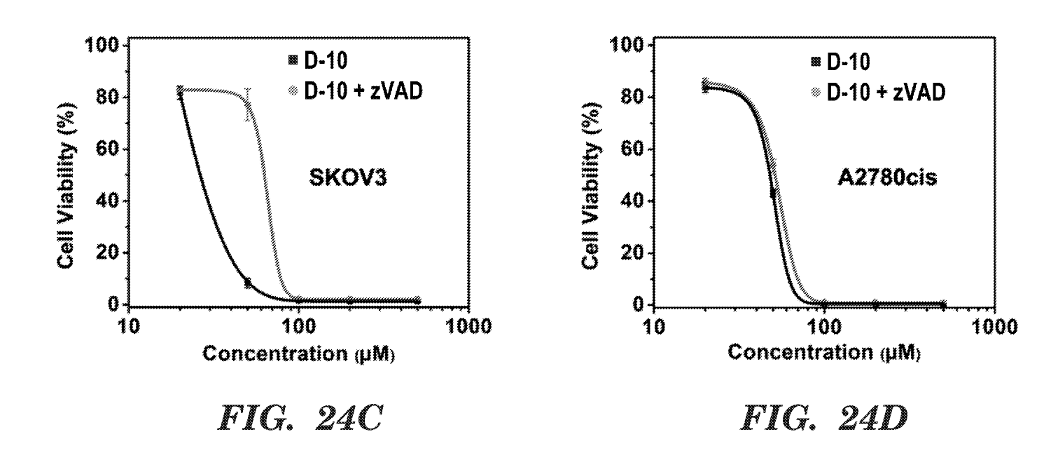

FIG. 6A-B are TEM images, optical images, and analytical HPLC spectra of the solution or hydrogel after the addition of esterase (1 U/ml) for 1 min (FIG. 6A) and 24 hours (FIG. 6B).

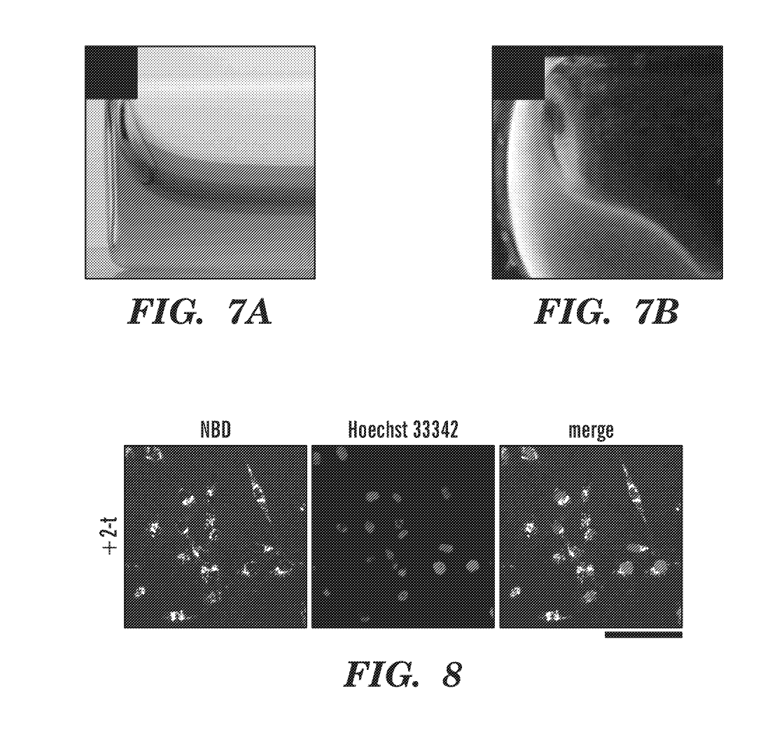

FIG. 7A-B are optical images of the solution of 1 (1.0 wt %) (FIG. 7A) and hydrogel formed by treating the solution of 1 with esterase (1 U/ml) (FIG. 7B) under the excitation of a hand-held UV lamp (λₑₓ=365 nm).

FIG. 8 is a panel of fluorescent confocal microscopy images showing the fluorescence emission in HeLa cells with the treatment of 2-t at the concentration of 200 μM in culture medium for 24 hours and stained with Hoechst 33342 (nuclei).

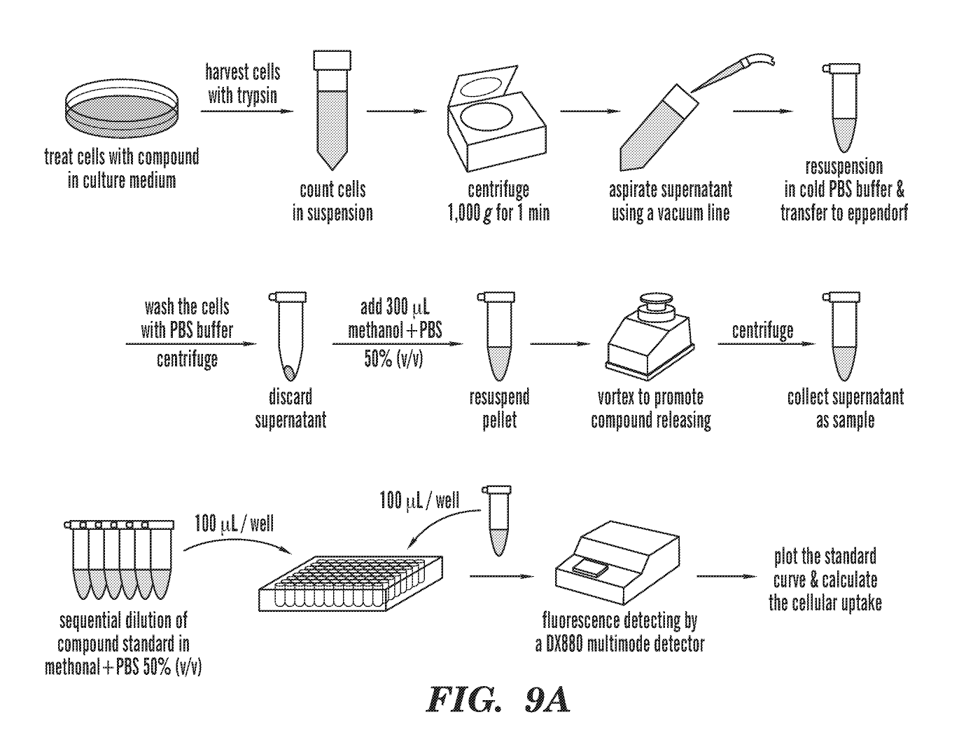

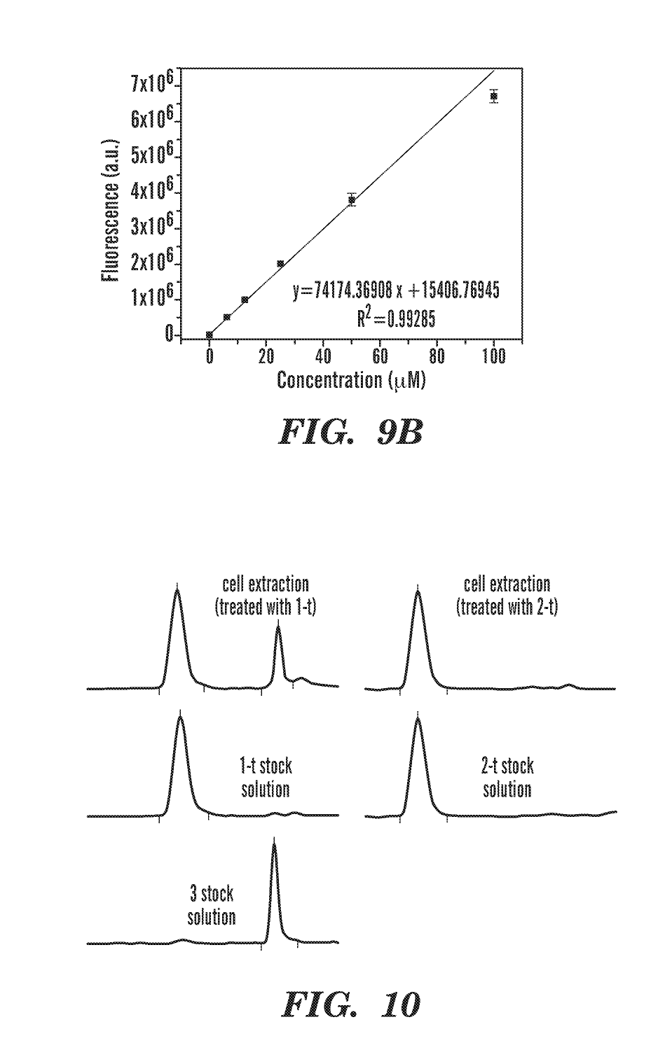

FIG. 9A is a schematic illustration of the procedure of cellular uptake measurement, and FIG. 9B is a standard curve.

FIG. 10 is series of cell extraction analyses by analytical HPLC, detected at λ=480 nm. The spectra shown are all in the same time scale.

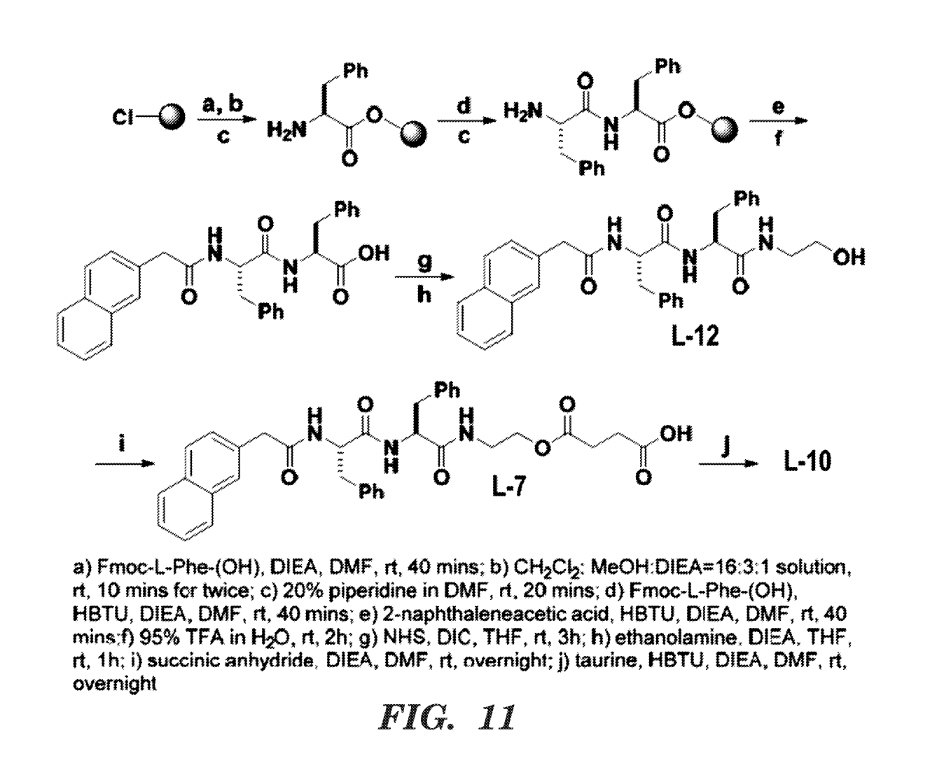

FIG. 11 shows general synthetic route for the precursor (L-10 as an example). The enzymatically activated form, L-12, is an intermediate in the synthetic scheme. D-10 is similarly prepared using Fmoc-D-Phe-OH rather than Fmoc-L-Phe-OH.

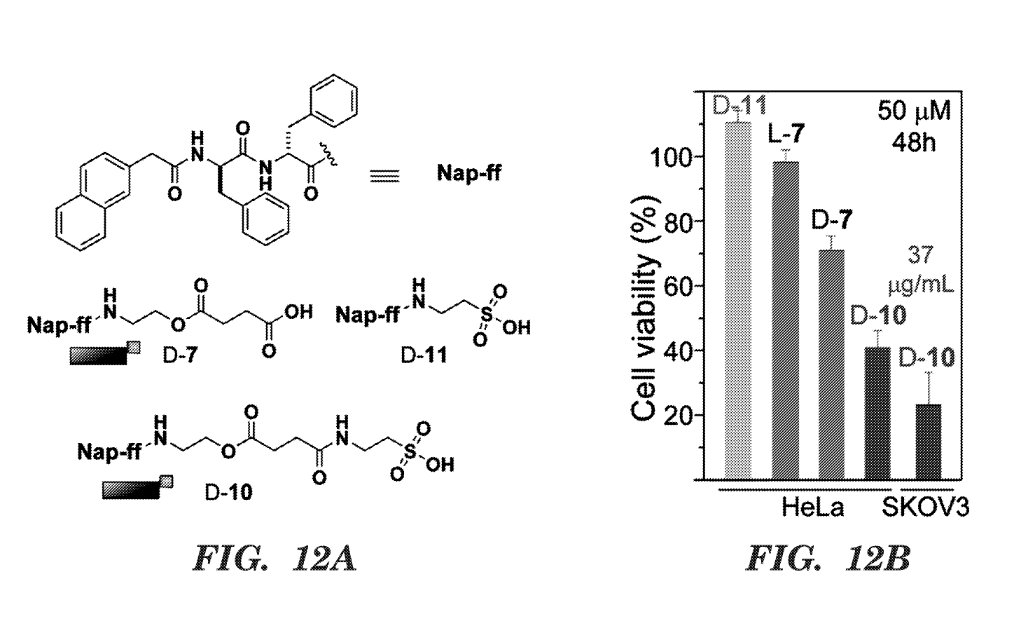

FIGS. 12A-B illustrate the increased anticancer activity of nanofibers of D-peptides compared to their corresponding L peptides. FIG. 12A shows structures of the substrates of esterases, including 2-(napthalen-2-yl)-aceytl-F_{D}F_{D} (“Nap-ff”) bearing a C-terminal ester-linked proprionic acid group (D-7); its corresponding L enantiomer, L-7; and Nap-ff bearing a C-terminal ester-linked taurine conjugate, D-10. The compound Nap-ff bearing a C-terminal amide-linked taurine residue, designated D-11, served as a control since it lacks the esterase substrate. FIG. 12B is a graph illustrating cell viabilities after the cells (HeLa or SKOV3) were treated by the indicated molecules for 48 hours (50 μM for HeLa cells, 37 μg/mL for SKOV3 cells).

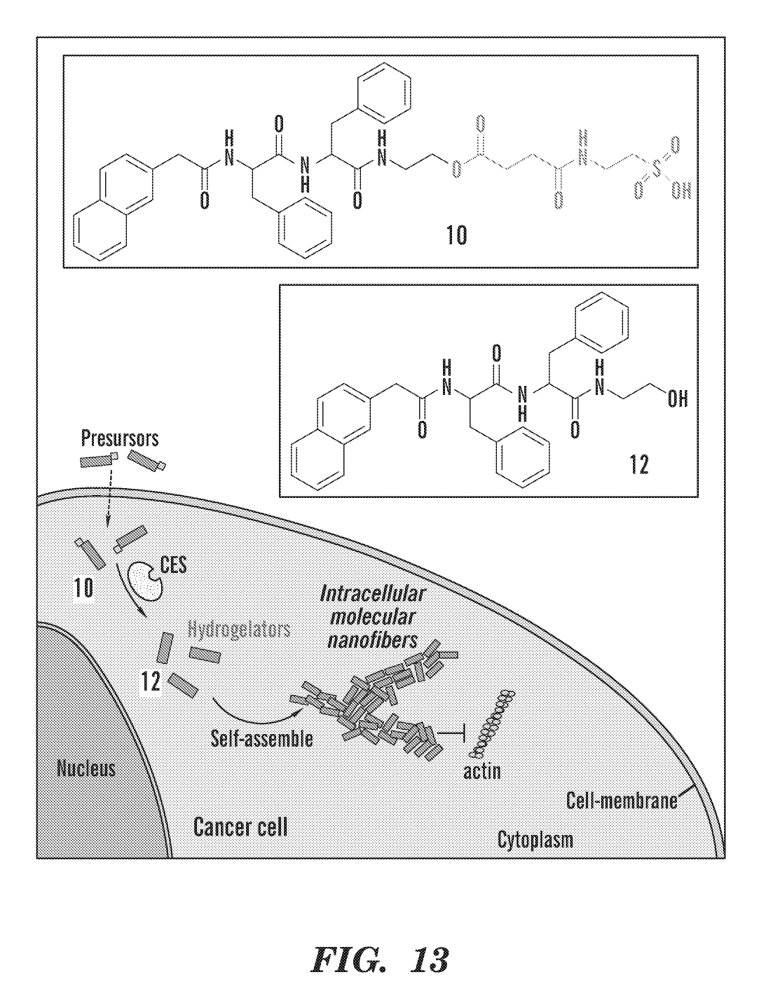

FIG. 13 illustrates the enzymatic transformation of the precursor (generically 10; hereinafter also L-10 and D-10) as a substrate of carboxylesterase (CES) to the corresponding hydrogelator (generically 12; hereinafter also L-12 and D-12) for intracellular self-assembly within a cancer cell. The self-assembled nanofibers inhibit actin filament formation and, thus, cellular processes that involve the same.

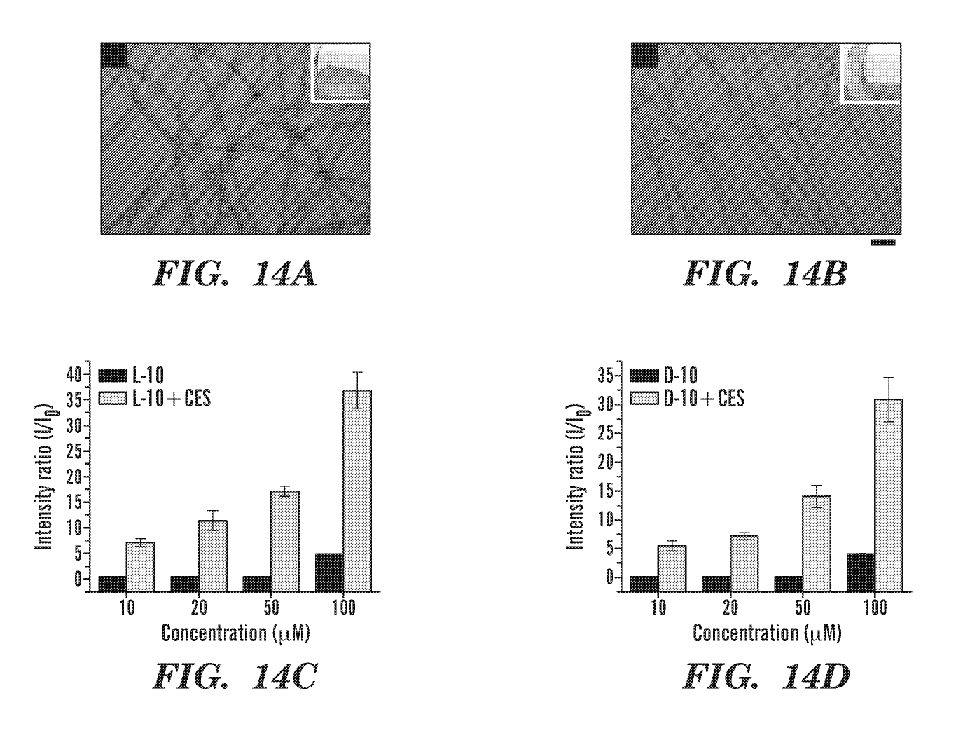

FIGS. 14A-D are TEM images of the hydrogels and graphs showing signal intensity ratio of static light scattering (SLS) of the solution of L-10 and D-10 at various concentrations. TEM images of the hydrogels (inset: optical images) formed by the addition of CES (2 U/mL) to the solution of L-10 (FIG. 14A) or D-10 (FIG. 14B) at the concentration of 0.4 wt % in PBS buffer (Scale bar: 100 nm). The signal intensity ratio of static light scattering (SLS) of the solution of L-10 (FIG. 14C) or D-10 (FIG. 14D) at concentrations from 10 to 100 μM before (black bar) and after (gray bar) being treated CES (2 U/mL) for three hours.

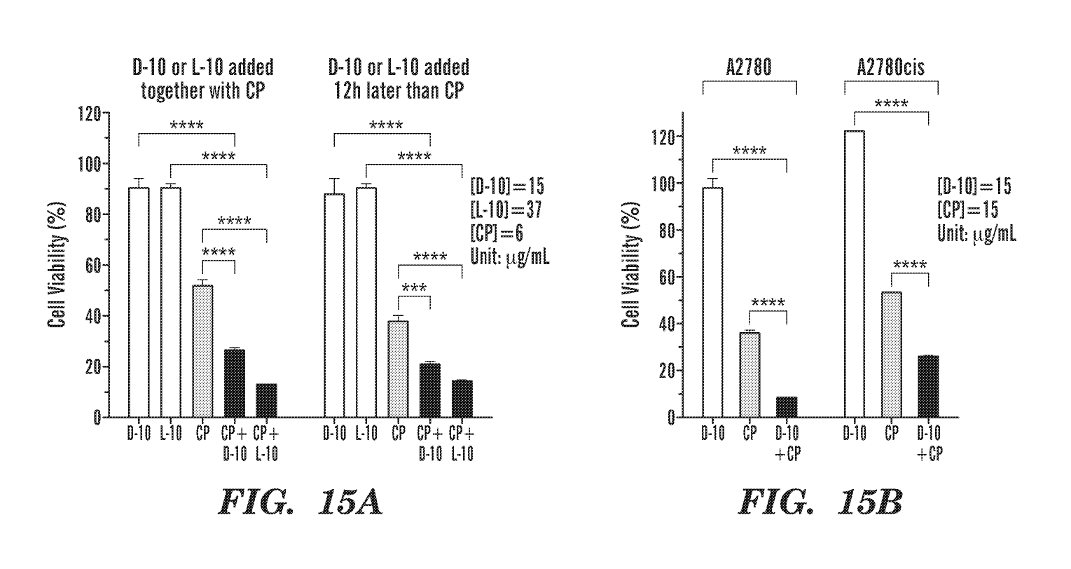

FIGS. 15A-B are graphs showing cell viability of SKOV3 ovarian cancer cells incubated with the precursors with and without cisplatin (CP). FIG. 15A shows the cell viability of SKOV3 cells incubated with the precursors D-10 or L-10 alone, or in combination with CP for 72 hours. FIG. 15B shows the cell viability of A2780 cells and A2780cis cells incubated with the precursor D-10 alone or in combination with CP for 72 hours (***=p<0.001, ****=p<0.0001).

FIG. 16 are fluorescence images of SKOV3 cells stained with Alexa Fluor 633 Phalloidin (F-actin) and Hoechst (nuclei) after treatment of D-10 at concentration of 20 μM for 20 hours (upper) or without the treatment of D-10 (bottom). Scale bars: left=20 right=10 μm.

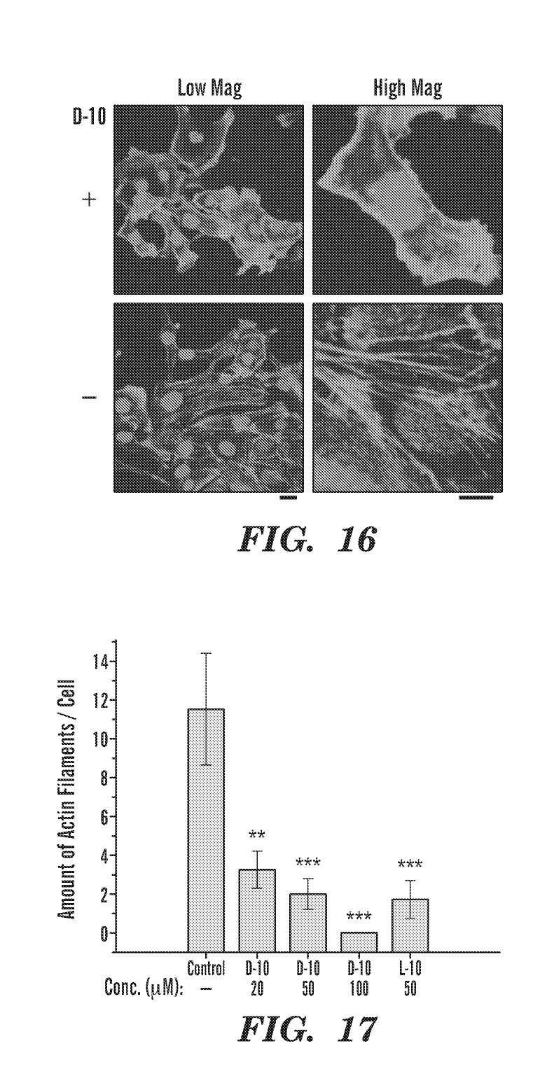

FIG. 17 is a graph showing the amount of actin filaments (longer than 5 μm) in SKOV3 cells after being treated by medium containing 0 μM, 20 μM, 50 μM, and 100 μM of D-10 or 50 μM of L-10 (**=p<0.01, ***=p<0.001 versus control).

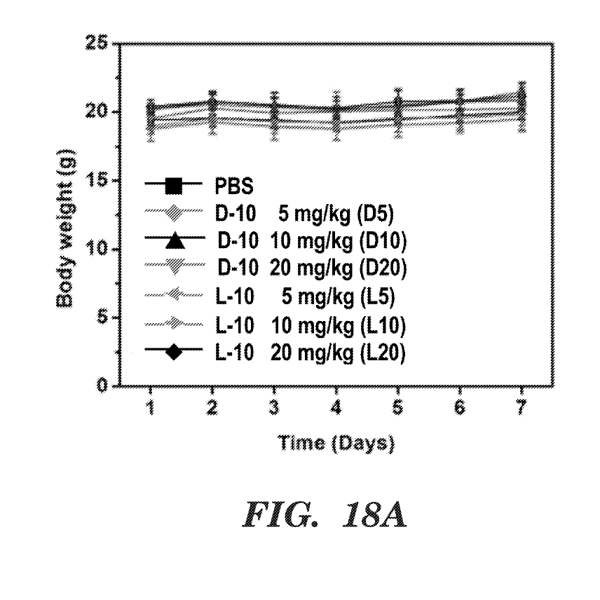

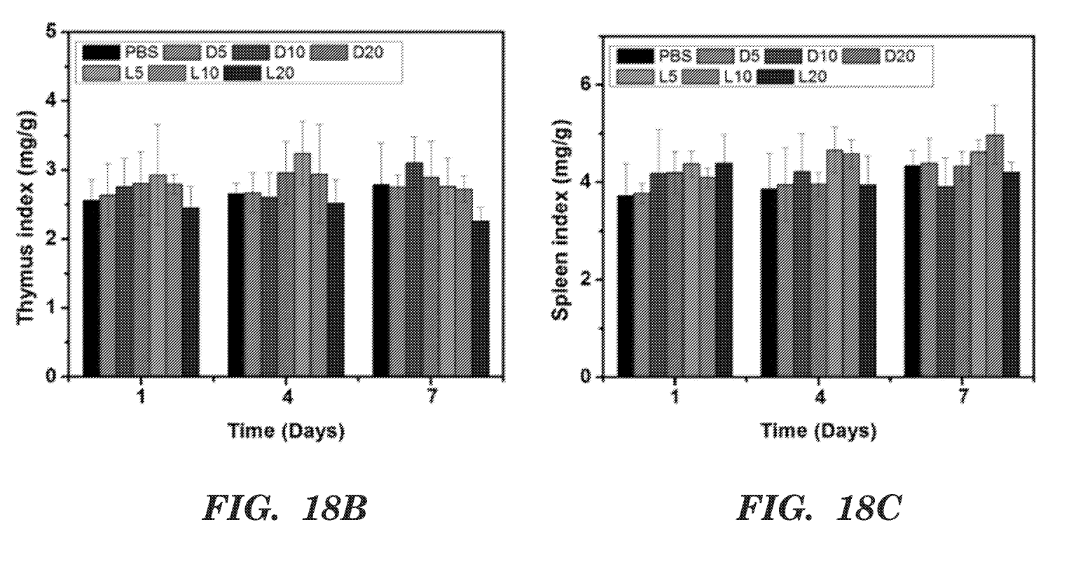

FIGS. 18A-C show body weight curves of mice after injection of D-10 or L-10 at 5 mg/kg, 10 mg/kg, and 20 mg/kg or PBS buffer (data are shown as mean±SD of body weight (n=4)) (FIG. 18A), as well as the thymus index (FIG. 18B) and spleen index (FIG. 18C) from the treated mice.

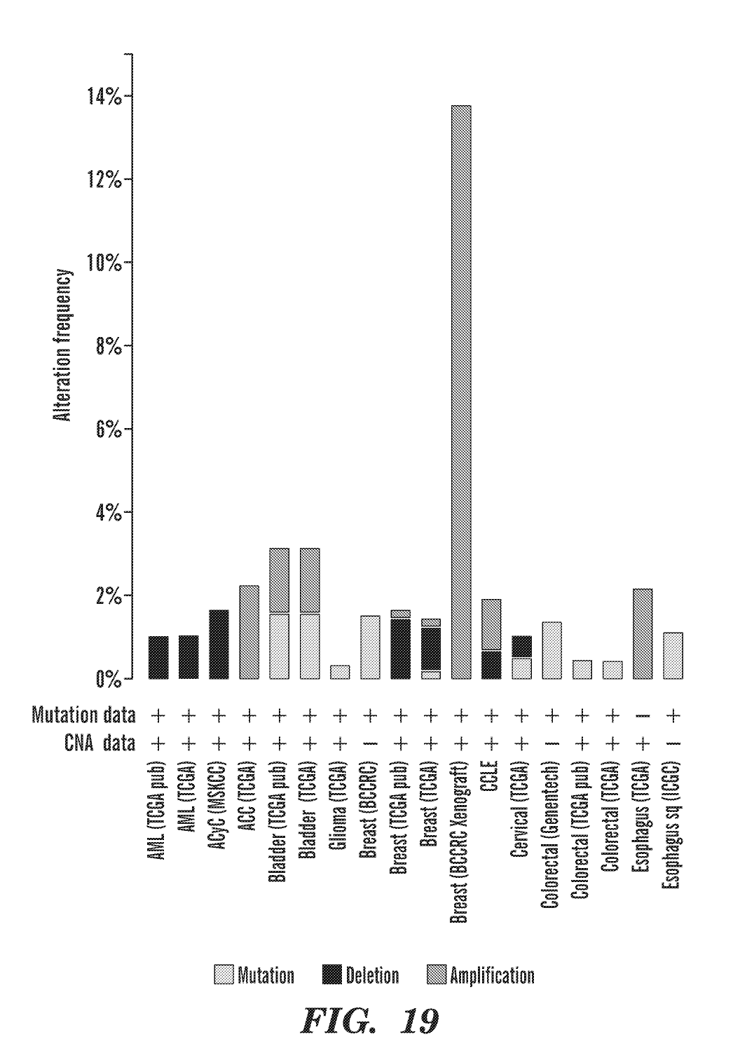

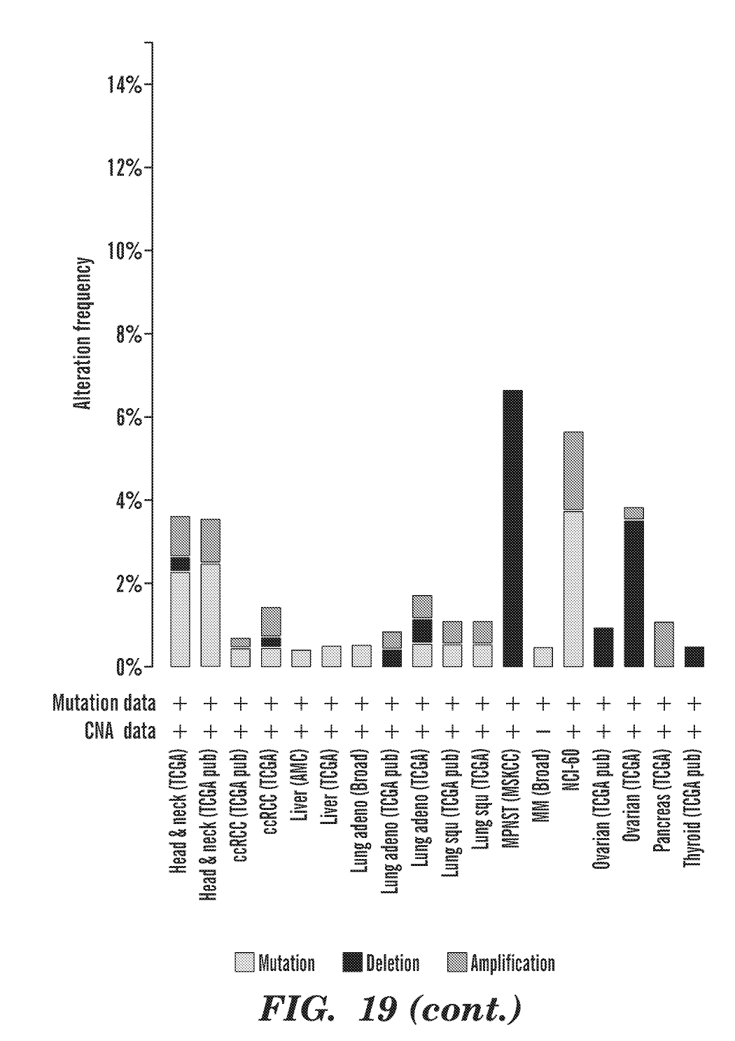

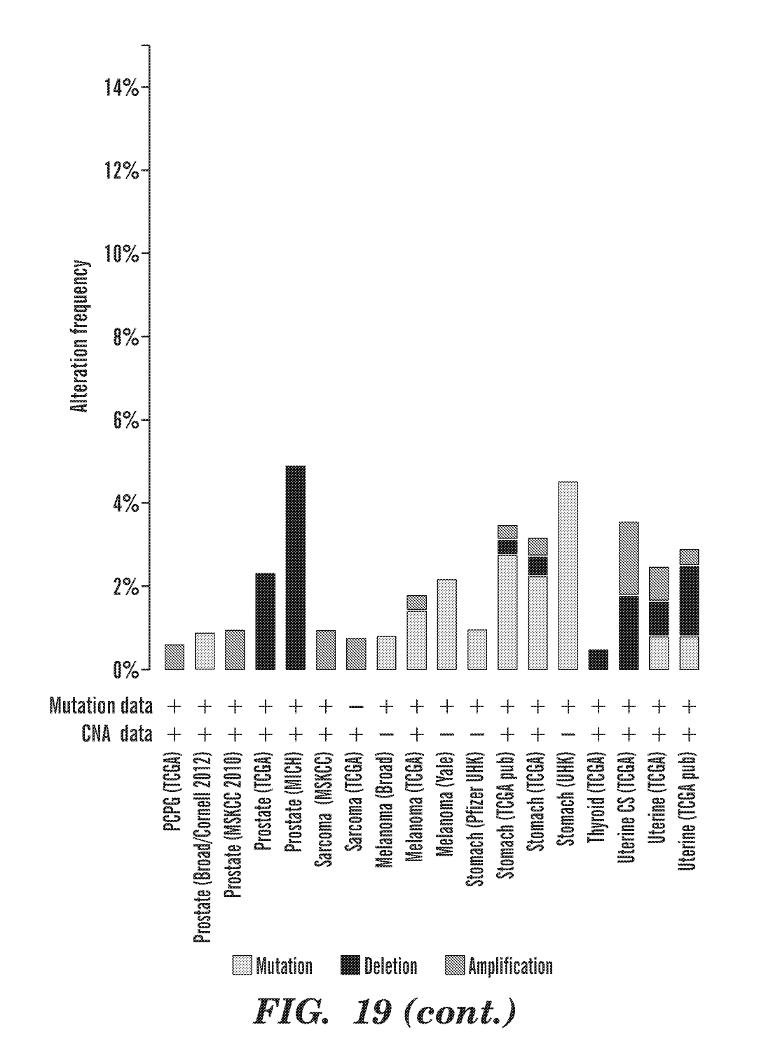

FIG. 19 provides a cross-cancer alteration summary for CES from different databases and cancer types: the alteration frequency profile of CES includes mutation (dark gray), deletion (black), amplification (medium gray), and multiple alterations (light gray). While CES expression differs dramatically throughout various cancers and organs, many cancer types.

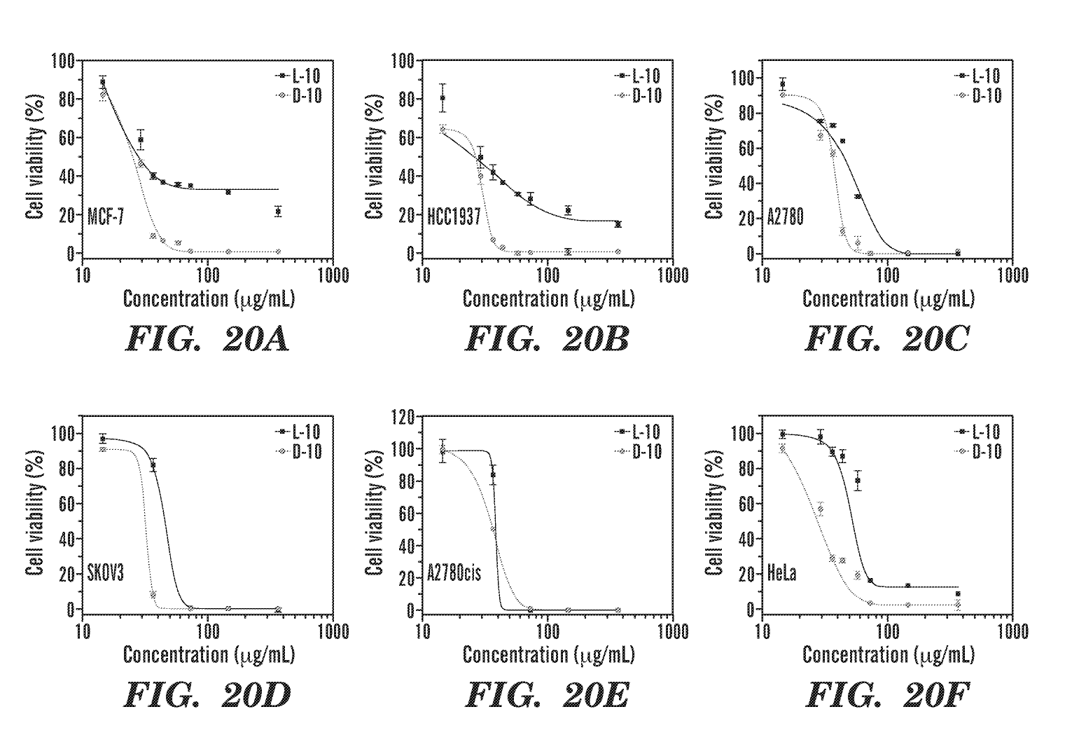

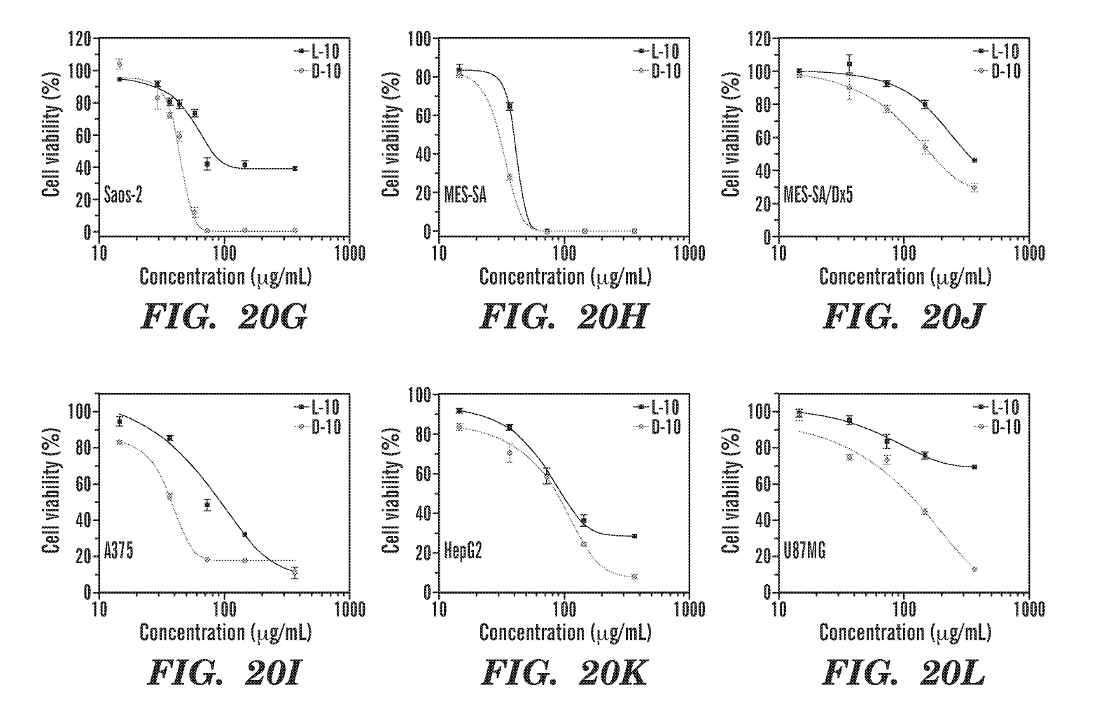

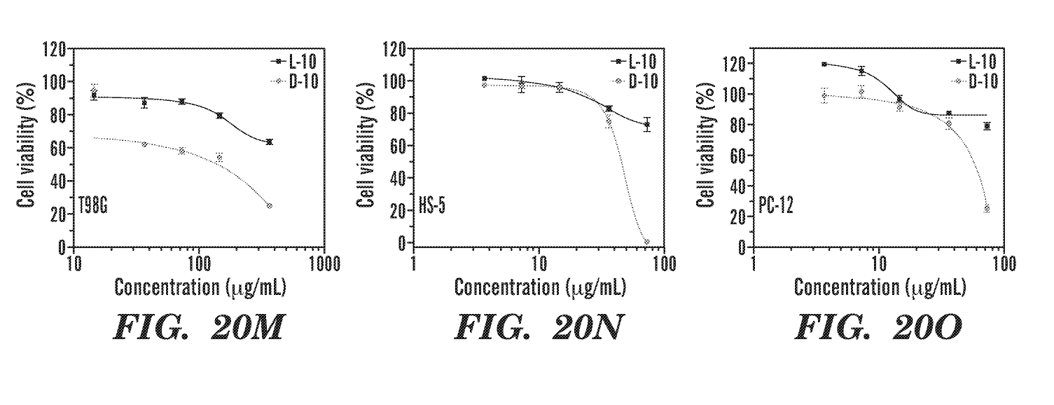

FIGS. 20A-O illustrate the cell viabilities of multiple cell lines incubated with L-10 (black curve) or D-10 (grey curve) for 72 hours. Cell viabilities of the following cell lines were tested: a triple negative breast cancer cell line (HCC1937), a breast cancer cell line (MCF-7), a drug sensitive ovarian cancer cell line (A2780), two drug resistant ovarian cancer cell lines (A2780cis and SKOV3), an adenocarcinoma cell line (HeLa), an osteosarcoma cell line (Saos-2), a drug sensitive sarcoma cell line (MES-SA), a drug resistant sarcoma cell line (MES-SA/D×5), a melanoma cancer cell line (A375), a hepatocellular carcinoma cell line (HepG2), two glioblastoma cell lines (U87MG and T98G), a stromal cell line (HS-5) and a neuronal cell line (PC-12). 10⁴ cells/well were initially seeded in a 96 well plate.

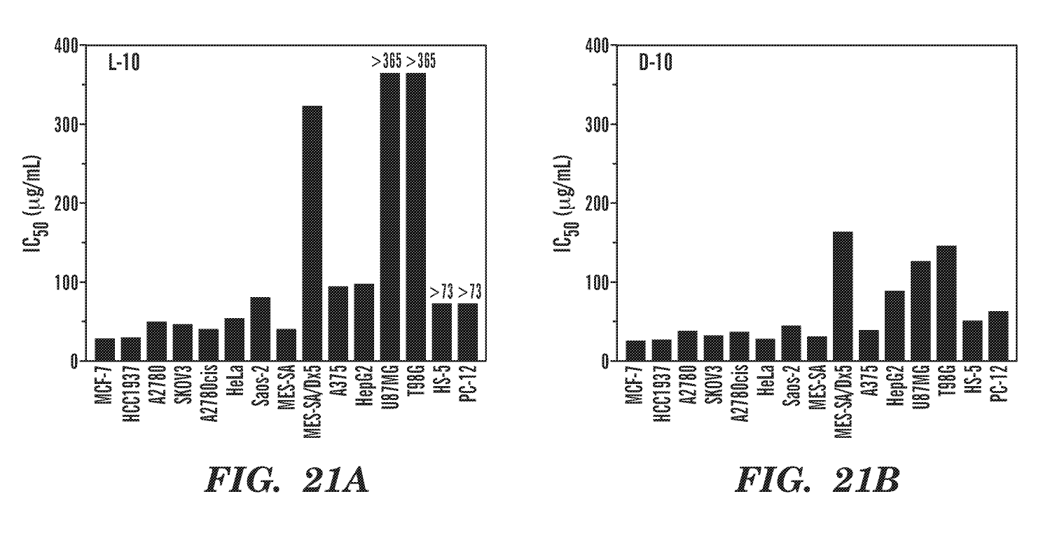

FIG. 21A-B summarize IC₅₀ values of L-10 and D-10, respectively, on multiple cell lines on the third day.

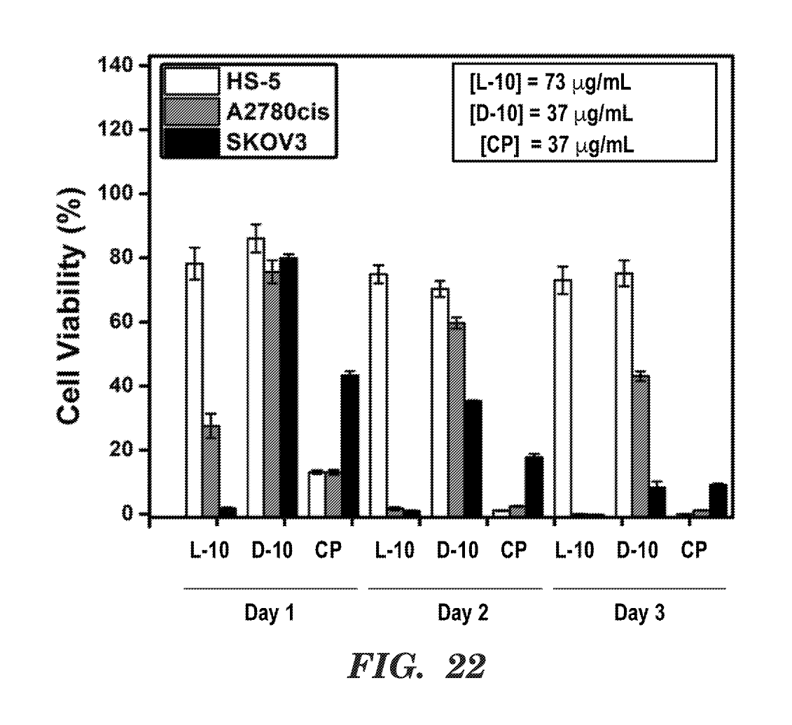

FIG. 22 illustrates cell viabilities of the stromal cells (HS-5) and ovarian cancer cells (A2780cis and SKOV3) (10⁴ cells/well were initially seeded in a 96 well plate) incubated with the precursor L-10 (73 μg/mL), D-10 (37 μg/mL), or cisplatin (37 μg/mL) for 3 days.

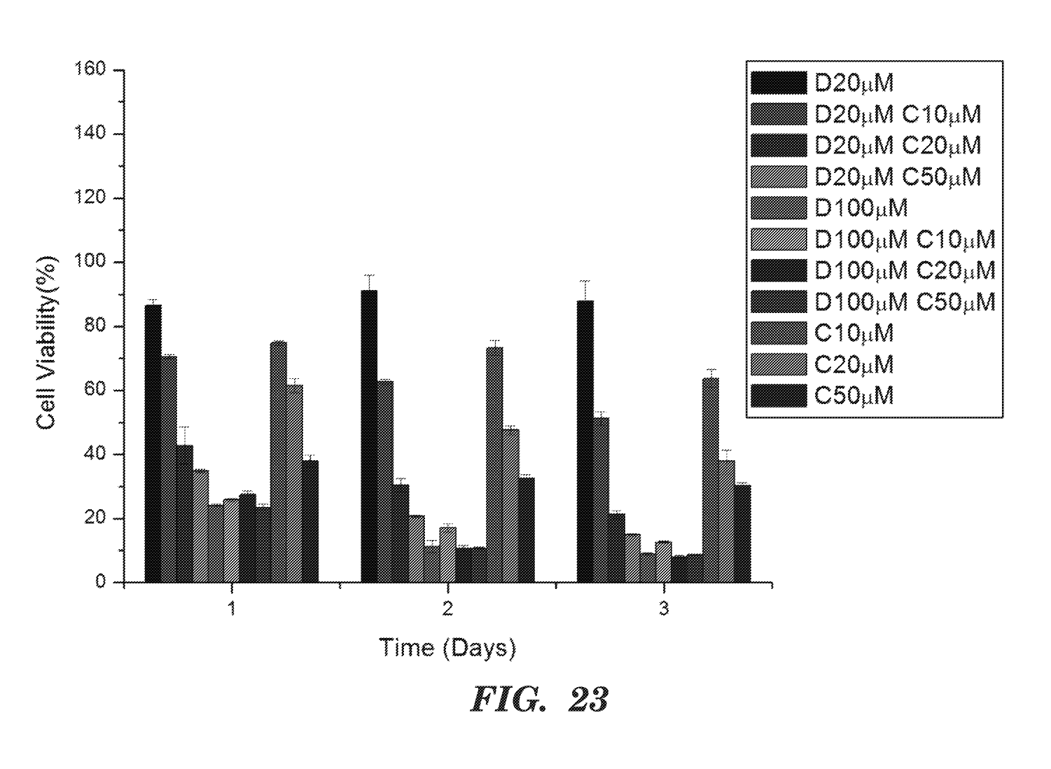

FIG. 23 is a graph illustrating viabilities of SKOV3 ovarian cancer cells (10⁴ cells/well were initially seeded in a 96 well plate) incubated with 20 or 100 μM D-10, alone or in combination with 10, 20 or 50 μM cisplatin for 3 days. SKOV3 cells exposed to these same concentrations of cisplatin for 3 days are also shown as control. The inset legend identifies the treatments from top-to-bottom, which correspond to the bar graphs from left-to-right.

FIG. 24A-D illustrate cell viability of SKOV3 cells and A2780cis cells incubated with L-10 alone or L-10+zVAD (45 μM) (FIGS. 24A-B), and D-10 alone or D-10+zVAD (45 μM) (FIGS. 24C-D) for 72 hours. zVAD is benzyloxycarbonyl-val-ala-asp (ome) fluoromethylketone (or z-vad-Fmk).

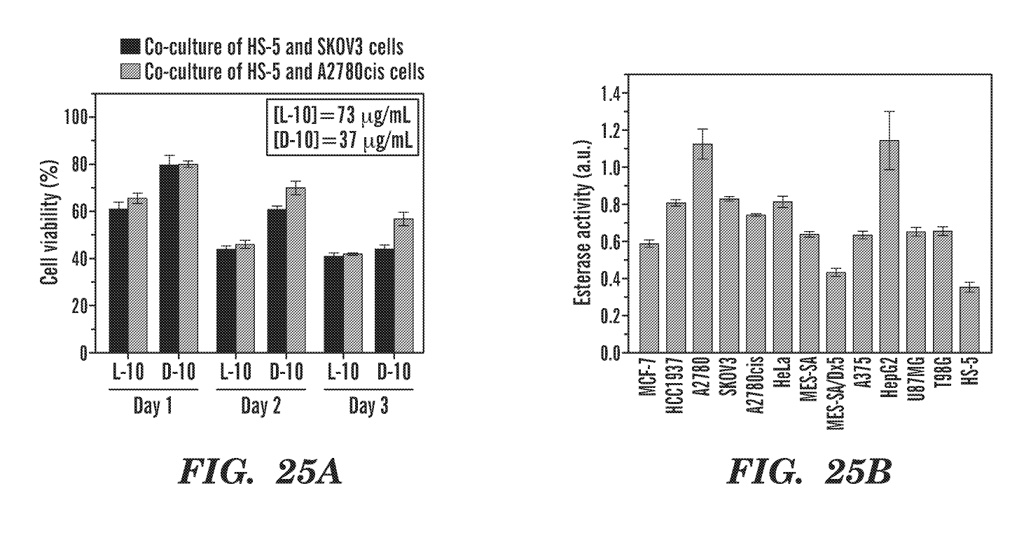

FIG. 25A illustrates cell viabilities of the co-cultured SKOV3/HS-5 cells and A2780cis/HS-5 cells incubated with the precursor L-10 (73 μg/mL) or D-10 (37 μg/mL) for 3 days. 5000 of each co-cultured cells were initially seeded in a 96 well plate. FIG. 25B illustrates esterase activities in multiple cell lines.

### DETAILED DESCRIPTION OF THE INVENTION

One aspect of the present invention relates to a peptide that in its state of administration is innocuous to normal cells, but upon exposure to cellular enzymes, particularly endoenzymes expressed by cancer cells, causes peptide self-assembly to form nanofibers and hydrogels internally of the cells. The self-assembly to form nanofibers and hydrogels can be carried out in vivo and ex vivo. These nanofibers and hydrogels have the capacity to physically alter the cells and their interactions with the cellular microenvironment. Use of these peptides, and compositions containing the same, is contemplated for the treatment of patients for cancerous or precancerous conditions, as well as for inhibiting cancer cell migration, inhibiting cancer cell survival, or inhibiting cancer cell growth. Use of the peptides that include a fluorophore conjugate is contemplated for cell imaging studies.

According to one embodiment, the peptide comprises a plurality of amino acid residues and an enzymatically cleavable moiety comprising a taurine or hypotaurine residue, the enzymatically cleavable-moiety being linked to the peptide via covalent bond, wherein the peptide is capable of self-assembly to form nanofibrils in the presence of an enzyme that hydrolyzes the enzymatically cleavable-moiety. The plurality of amino acid residues promote peptide self-assembly following enzymatic cleavage of the cleavable moiety. The taurine or hypotaurine residue promotes cellular uptake of the peptide prior to its enzymatic activation.

The amino acid residues that form the peptide can be any naturally occurring or non-naturally occurring amino acid, but preferably the peptide includes one or more aromatic amino acids. Aromatic amino acids used in the peptides of the present invention include, without limitation, any one or more of phenylalanine, phenylalanine derivatives, tyrosine, tyrosine derivatives, tryptophan, and tryptophan derivatives. Any known or hereinafter developed phenylalanine derivatives, tyrosine derivatives, or tryptophan derivatives can be used in the present invention, as long as the derivatives facilitate self-assembly of the nanofibers. Exemplary derivatives of these amino acids include the addition of one or more ring substituents.

The peptides can include all D-amino acids, all L-amino acids, or a mixture of L-amino acids and D-amino acids. In preferred embodiments, the peptide includes only D-amino acids or a mixture of D-amino acids and L-amino acids where the D-amino acid content is greater than 50%, 60%, 70%, 80%, 90%, or 95%.

As a consequence of utilizing entirely D-amino acids or a high proportion of D-amino acids, it is possible to render the peptide protease resistant, e.g., resistant to proteinase K digestion.

In certain embodiments, the peptide can include one or more amino acids whose side-chain is easily conjugated to, e.g., a fluorophore, a cytotoxic agent such as a chemotherapeutic agent, an antiangiogenic agent, or an immunomodulating agent, an antigen, or a thermoablative nanoparticle. Numerous examples of each of these categories are well known in the art.

Exemplary amino acids that can be derivatized include lysine or arginine, whose terminal amino group of its side chain is reactive in conjugation procedures of the type described in the (see Gao et al., Nat. Commun 3:1033 (2012), showing Lys-conjugated NBD, which is hereby incorporated by reference in its entirety; Gao et al., J. Am. Chem. Soc. 131(38):13576-13577 (2009), showing Lys-conjugated paclitaxel, which is hereby incorporated by reference in its entirety). Other conjugation protocols can be utilized with other amino acids, including aspartic and glutamic acid whose carboxylic acid groups are reactive in known conjugation procedures. Similarly, cysteine and cysteine derivatives can be used to form disulfide bonds during conjugation procedures. Allyl glycine can also be used in this regard.

The peptides of the present invention can have any length that is sufficient to allow for self-assembly once the enzyme (preferably an endoenzyme having hydrolase activity) cleaves the enzymatically cleavable-moiety covalently attached to the peptide. This includes peptides up to about 35 amino acids, up to about 30 amino acids, up to about 25 amino acids, up to about 20 amino acids, up to about 15 amino acids, or up to about 10 amino acids. In certain embodiments, the peptides contain from 2 to 10 amino acids, such as between 3 to 10 amino acids.

In certain embodiments, the peptide contains about 10 percent up to about 100 percent of aromatic amino acid residues.

The enzymatically cleavable moiety containing a taurine residue (—NH⁻—CH₂—CH₂—S(O₂)—OH) or a hypotaurine residue (—NH⁻—CH₂—CH₂—S(O)—OH) is covalently linked to the peptide via a covalent bond, typically though not exclusively a peptide bond formed at the C-terminal end of the peptide. In the enzymatically cleavable moiety, there is provided a bond which is cleavable in the presence of an enzyme that hydrolyzes the cleavable bond. In certain embodiments the enzyme is an esterase, preferably an esterase that is expressed internally of cancer cells. Examples of bonds that can be cleaved by an esterase include, without limitation, esters, carbonates, thiocarbonates, carbamates, carboxylates, and diacyl anhydrides. In other embodiments, the enzyme is a protease and the peptide includes an amide bond that can be cleaved by a protease.

Exemplary enzymatically cleavable moieties containing taurine or hypotaurine include, without limitation:

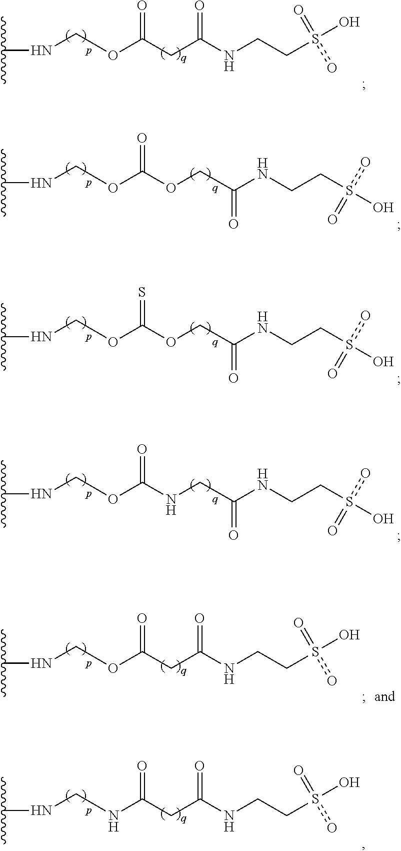

where p and q are independently integers from 1 to 5, or 1 to 3.

In each of the preceding embodiments, the peptide may optionally include an N-terminal amino acid that is capped by a capping moiety. The capping moiety preferably includes an acyl group due to the reaction of a carboxylic acid with the N-terminal amino group to form a peptide bond. These capping moieties can protect against enzymatic degradation of the peptide, promote self-assembly in the case where aromatic groups are present in the capping moiety, promote fluorescence of a hydrogel fiber or network, as well as afford improved cytotoxicity. In certain embodiments, the capping moiety is one that is hydrophobic.

The capping moiety may or may not include an aromatic group. Exemplary capping moieties include, without limitation, alkylacyls such as acetyl, proprionyl, or fatty acid derivatives, arylacyls such as 2-naphthalacetyl or 3-((7-nitrobenzo(c)-1,2,5-oxadiazol-4-yl)amino)proprionyl (“NBD”), or an acylated nucleoside or nucleoside analog. NBD is a fluorophore and it can be used to track assembly of hydrogels, as discussed in the accompanying examples. Alternatively, a drug (e.g., cytotoxic drug) or other agent can be covalently linked to the N-terminus of the peptide. Exemplary drugs for N-terminal conjugation include, without limitation, doxorubicin (Zhang et al., “Cellular Uptake and Cytotoxicity of Drug-Peptide Conjugates Regulated by Conjugation Site,” Bioconjug Chem. 24(4):604-613 (2013), which is hereby incorporated by reference in its entirety), daunomycin (Varga, “Hormone-drug conjugates,” Methods in Enzymology 112:259-269 (1985), which is hereby incorporated by reference in its entirety), methotrexate (Radulovic et al., “Cytotoxic analog of somatostatin containing methotrexate inhibits growth of MIA PaCa-2 human pancreatic cancer xenografts in nude mice,” Cancer Letters 62:263-271 (1992), which is hereby incorporated by reference in its entirety), and paclitaxel (see Gao et al., J. Am. Chem. Soc. 131(38):13576-13577 (2009), showing Lys-conjugated paclitaxel using succinic anhydride and NHS succinate, which is hereby incorporated by reference in its entirety). Cytotoxic nucleoside analogs or nucleobases that can be incorporated at the N-terminal end of the peptide include, without limitation, vidarabine, cytarabine, gemcitabine, fludarabine, cladribine, pentostatin, 6-mercaptopurine, thioguanine, and fluorouracil,

Exemplary peptides of the present invention include, without limitation,

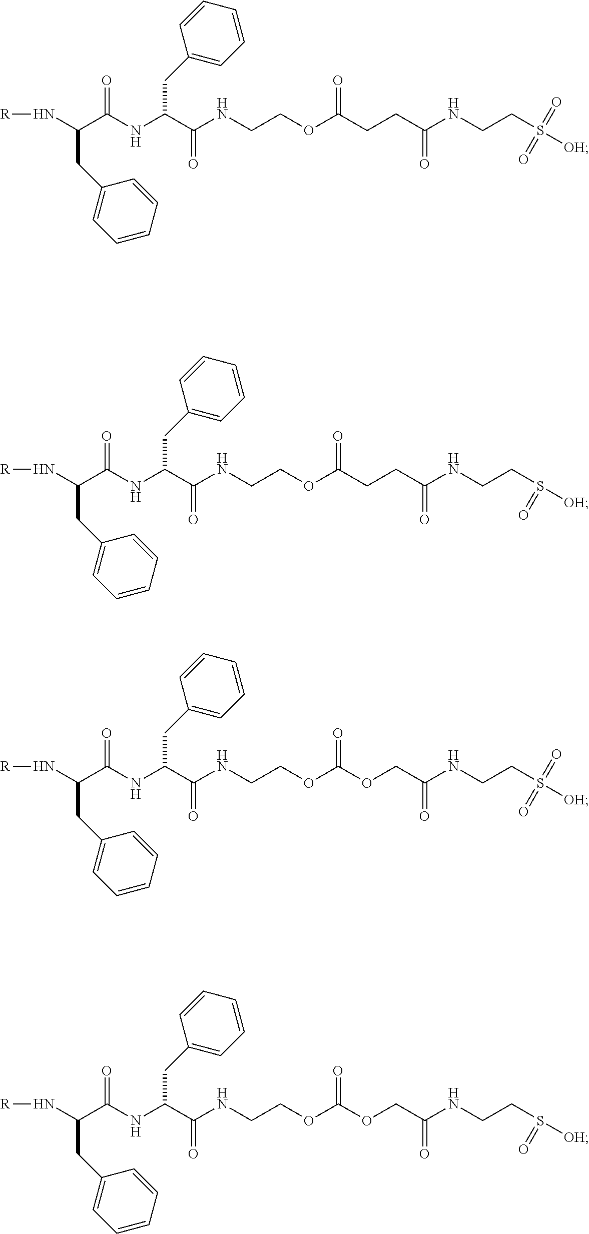

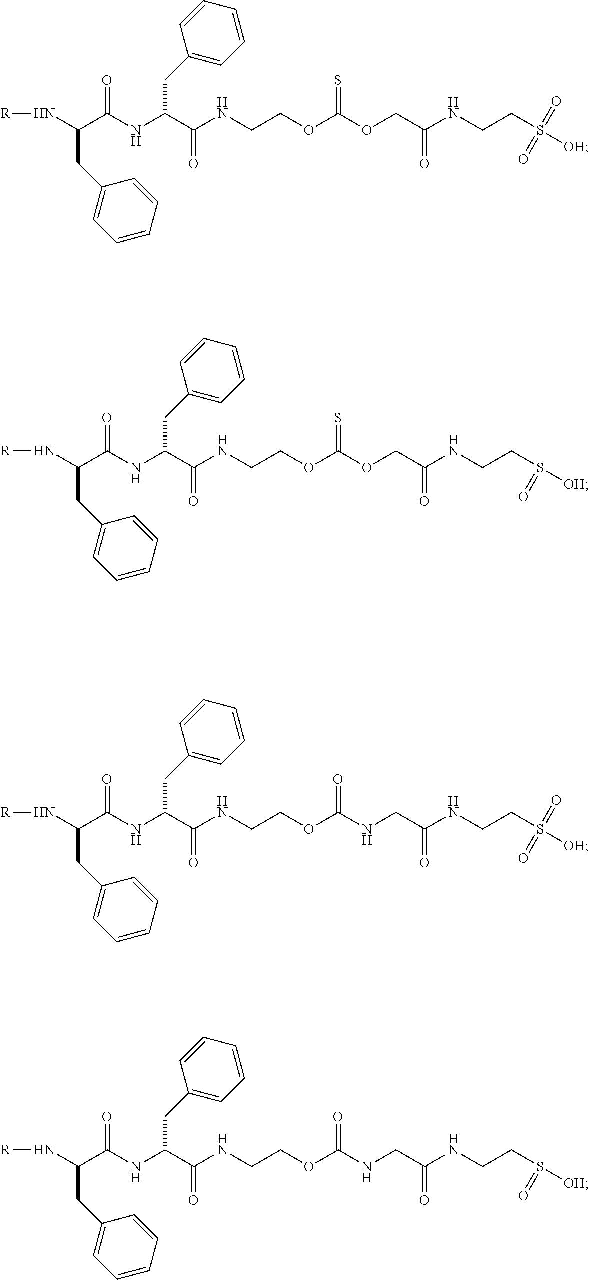

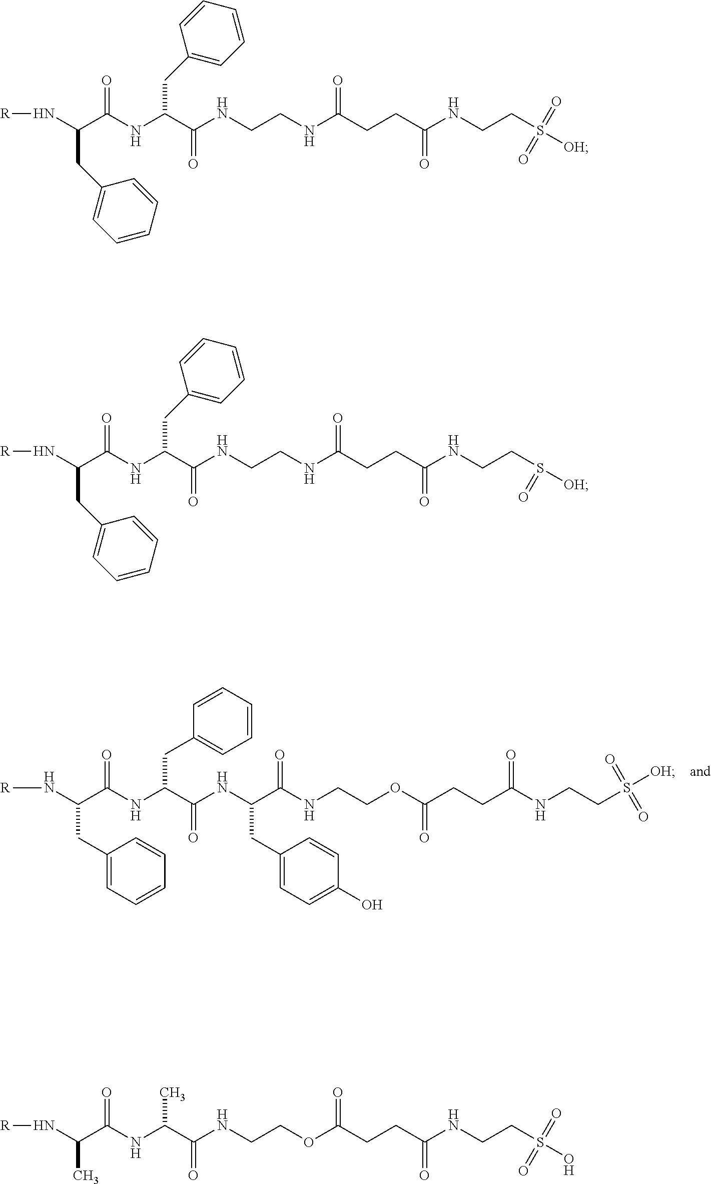

where R can be H or an N-terminal capping moiety of the type described above. Exemplary fluorophores at the N-terminus include, without limitation,

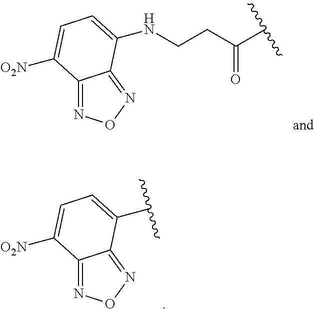 [SMILES: *C(=O)CCNc1ccc([N+](=O)[O-])c2nonc12 ; *c1ccc([N+](=O)[O-])c2nonc12]

Exemplary aromatic ring structures at the N-terminus include arylacyl groups such as phenylacetyl and napthylacetyl.

Compounds of the present invention can be prepared according to the procedures shown in FIGS. 1A-B, FIG. 11, and Schemes 1-4 below.

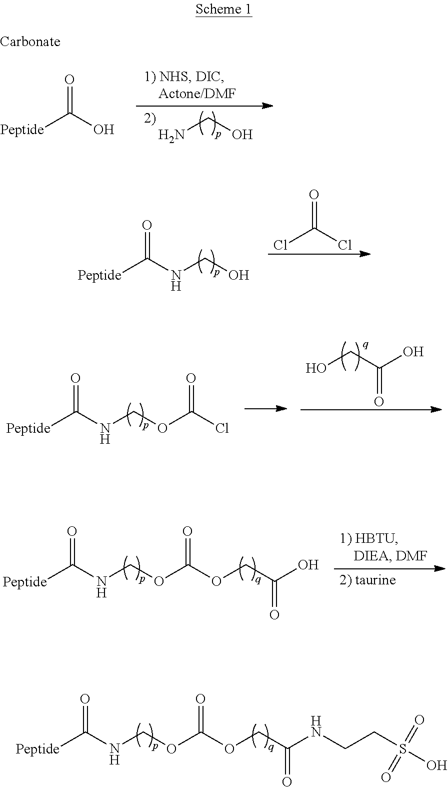

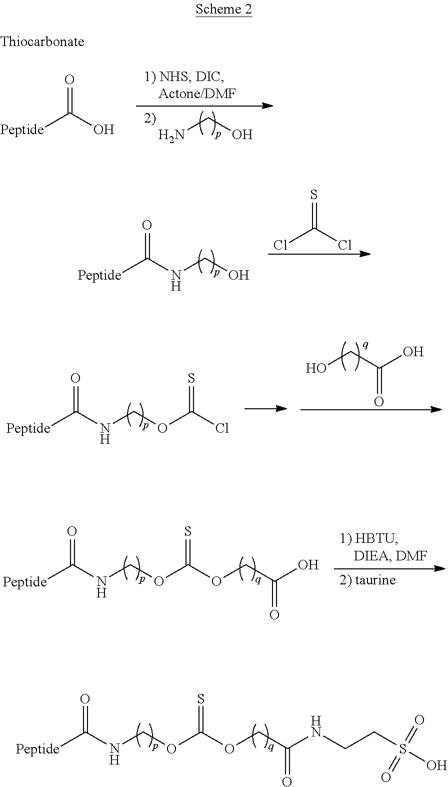

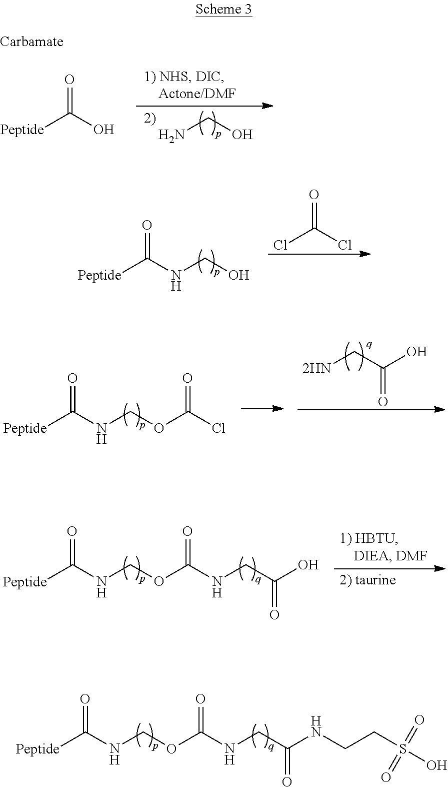

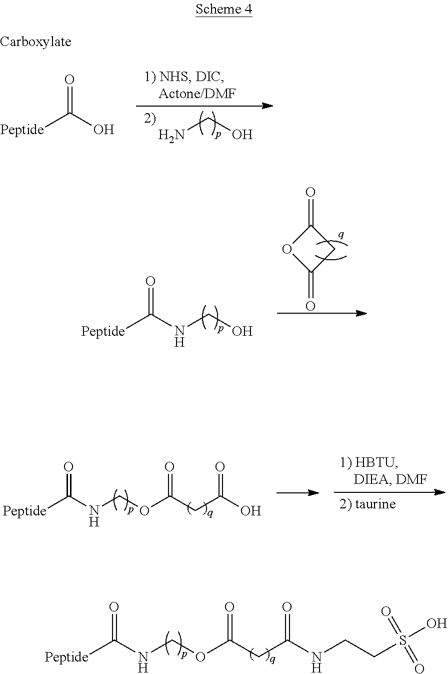

The peptides of the present invention can be synthesized using standard peptide synthesis operations. These include both FMOC (9-Fluorenylmethyloxy-carbonyl) and tBoc (tert-Butyl oxy carbonyl) synthesis protocols that can be carried out on automated solid phase peptide synthesis instruments including, without limitation, the Applied Biosystems 431A, 433A synthesizers and Peptide Technologies Symphony or large scale Sonata or CEM Liberty automated solid phase peptide synthesizers. This can be followed with standard HPLC purification to achieve a purified peptide product.

Where N-terminal capping groups or C-terminal groups are introduced, these can be introduced using standard peptide synthesis operations as described above. For example, carboxylic acid containing precursors can be coupled by peptide bond to the N-terminus of the peptide, and amino containing precursors can be coupled by peptide bond to the C-terminus of the peptide.

Introduction of functional groups to the peptide can also be achieved by coupling via side chains of amino acids, including the amino group of lysine, the guanidine group of arginine, the thiol group of cysteine, or the carboxylic acid group of glutamic acid or aspartic acid.

In general, amino groups present in lysine side chains, as well as the N-terminal amino group, can be reacted with reagents possessing amine-reactive functional groups using known reaction schemes. Exemplary amine-reactive functional groups include, without limitation, activated esters, isothiocyanates, and carboxylic acids. Reagents to be conjugated include those listed above. Examples of conjugating a chemotherapeutic agent (e.g., doxorubicin, daunorubicin, taxol) to a Lys sidechain are described in DeFeo-Jones et al., Nature Med. 6(11):1248-52 (2000), Schreier et al., PlosOne 9(4):e94041 (2014), Gao et al., J Am Chem Soc. 131:13576 (2009), each of which is hereby incorporated by reference in its entirety.

In general, guanidine groups present in arginine can be reacted with reagents possessing guanidine-reactive groups using known reaction schemes. Exemplary guanidine-reactive functional groups include, without limitation, NHS esters using gas phase synthesis (McGee et al., J. Am. Chem. Soc., 134 (28):11412-11414 (2012), which is hereby incorporated by reference in its entirety).

In general, thiol groups present in cysteine (or cysteine derivative) side chains can be reacted with reagents possessing thiol-reactive functional groups using known reaction schemes. Exemplary thiol-reactive functional groups include, without limitation, iodoacetamides, maleimides, and alkyl halides. Reagents to be conjugated include those listed above.

In general, carboxyl groups present in glutamic or aspartic acid side chains, or at the C-terminal amino acid residue, can be reacted with reagents possessing carboxyl-reactive functional groups using known reaction schemes. Exemplary carboxyl-reactive functional groups include, without limitation, amino groups, amines, bifunctional amino linkers. Reagents to be conjugated include those listed above.

In each of the types of modifications described above, it should be appreciated that the conjugate can be directly linked via the functional groups of the peptide and the reagent to be conjugated, or via a bifunctional linker that reacts with both the peptide functional groups and the functional groups on the reagent to be conjugated.

One example of conjugating the fluorophore NBD to the N-terminal end of a peptide is illustrated in FIG. 1A. Briefly, beta-alanine-derivatized NBD is first prepared so that the NBD fluorophore is functionalized with a carboxylic acid group suitable for reaction with the N-terminal amino acid. Once the peptide is prepared, the functionalized NBD is reacted with the peptide using standard reagents, e.g., O-(benzotriazol-1-yl)-1,1,3,3-tetramethyluronium hexafluorophosphate (HBTU) or 1-[bis(dimethylamino)methylene]-1H-1,2,3-triazolo[4,5-b]pyridinium-3-oxid hexafluorophosphate (HATU) or an equivalent (see Han et al., “Recent Development of Peptide Coupling Reagents in Organic Synthesis,” Tetrahedron 60:2447-2467 (2004), which is hereby incorporated by reference in its entirety) in N,N-Diisopropylethylamine or its equivalent (see id.).

Once any modification of the side chains or N-terminal group is complete, the enzymatically cleavable moiety containing the (hypo)taurine residue can be coupled to the C-terminus of the peptide. According to one example, this is achieved using NHS/DIC and ethanolamine followed by succinic anhydride in DIEA to form an ester-containing moiety bearing a reactive carboxylic acid group (e.g., compound 1 in FIG. 1A), which is then reacted with taurine using standard reagents of the type described above (see Han et al., “Recent Development of Peptide Coupling Reagents in Organic Synthesis,” Tetrahedron 60:2447-2467 (2004), which is hereby incorporated by reference in its entirety). Other approaches using different enzymatically cleavable moieties are illustrated in Schemes 1-4 above.

Once the peptides of the present invention are synthesized, they are preferably purified (preferably at least about 80% or 85% pure, more preferably at least about 90% or 95% pure, most preferably at least about 99% pure) by any suitable techniques. Exemplary purification techniques include, without limitation, gel filtration, ion exchange chromatography, hydrophobic interaction chromatography, affinity chromatography, reverse phase chromatography, and combinations thereof.

A further aspect of the present invention relates to pharmaceutical compositions that include a pharmaceutically acceptable carrier and a peptide of the present invention, which is present in an effective amount, preferably in a purified form.

In certain embodiments, more than one peptide can be provided. The peptides can similar in structure, but possess different conjugated agents as described above. In alternative embodiments, the peptides can be structurally distinct, including different structures that are nevertheless capable of self-assembly due to the structural compatibility of the aromatic amino acid residues in the different peptides.

By way of example, a peptide of the present invention lacking a conjugated chemotherapeutic agent can be combined with another peptide of the present invention that possesses a conjugated chemotherapeutic agent of the type described above. These can be provided in various ratios so as to facilitate an appropriate dosage of the enzymatically-activated, self-assembling peptides while also achieving a desired dose of the conjugated chemotherapeutic agent.

In certain embodiments, the carrier is an aqueous medium that is well tolerated for administration to an individual, typically a sterile isotonic aqueous buffer. Exemplary aqueous media include, without limitation, normal saline (about 0.9% NaCl), phosphate buffered saline (PBS), sterile water/distilled autoclaved water (DAW), as well as cell growth medium (e.g., MEM, with or without serum), aqueous solutions of dimethyl sulfoxide (DMSO), polyethylene glycol (PEG), and/or dextran (less than 6% per by weight.)

To improve patient tolerance to administration, the pharmaceutical composition preferably has a pH of about 6 to about 8, preferably about 6.5 to about 7.4. Typically, sodium hydroxide and hydrochloric acid are added as necessary to adjust the pH.

The pharmaceutical composition suitably includes a weak acid or salt as a buffering agent to maintain pH. Citric acid has the ability to chelate divalent cations and can thus also prevent oxidation, thereby serving two functions as both a buffering agent and an antioxidant stabilizing agent. Citric acid is typically used in the form of a sodium salt, typically 10-500 mM. Other weak acids or their salts can also be used.

The composition may also include solubilizing agents, preservatives, stabilizers, emulsifiers, and the like. A local anesthetic (e.g., lidocaine) may also be included in the compositions, particularly for injectable forms, to ease pain at the site of the injection.

Effective amounts of the peptide will depend on the nature of use, including the nature of the cancerous condition which is being treated, tumor volume and stage, and its location(s). By way of example only, suitable peptide concentrations may range from about 0.1 μM to about 10 mM, preferably about 1 μM to about 5 mM, about 10 μM to about 2 mM, or about 50 μM to about 1 mM. The volume of the composition administered, and thus, dosage of the peptide administered can be adjusted by one of skill in the art to achieve optimized results. In one embodiment, between 100 and about 800 μg can be administered per day, repeated daily or periodically (e.g., once every other day, once every third day, once weekly). This can be adjusted lower to identify the minimal effective dose, or tailored higher or lower according to the nature of the tumor to be treated.

In certain embodiments, the pharmaceutical composition can include, in addition to the peptide, one or more additional therapeutic agents. These additional therapeutic agents can include, without limitation, chemotherapeutic agents (including alkylating agents, platinum drugs, antimetabolites, anthracycline and non-anthracycline antitumor antibiotics, topoisomerase inhibitors, and mitotic inhibitors), corticosteroids and targeted cancer therapies (such as imatinib (Gleevec®), gefitinib (Iressa®), sunitinib (Sutent®) and bortezomib (Velcade®)), antiangiogenic agents, immunotherapeutic agents, and radiotherapeutic agents. These agents can be administered using conventional dosages or, alternatively, given the demonstrated non-additive effects of co-administering a chemotherapeutic agent with a peptide of the present invention, it is also contemplated that effective doses of these additional therapeutic agents can be further reduced (so as to minimize side effects) while also improving or maintaining the efficacy of the combination therapy as compared to the efficacy of the therapeutic agent alone. This is exemplified in the Examples using a combination therapy with cisplatin.

Also contemplated herein are therapeutic systems that include, as separate compositions, a first composition containing a peptide of the present invention in a suitable carrier, and a second composition containing an effective amount of one of the aforementioned additional therapeutic agents in a suitable carrier. These separate compositions can be co-administered according to a therapeutic protocol and dosing schedule.

Further aspects of the present invention relate to methods of forming a nanofibril network internally of cells generally, and more particularly to forming such a nanofibril network internally of cancer cells, delivering conjugated drugs (such as cytotoxic drugs) internally of cancer cells, as well as methods of treating a cancerous condition in a patient. Cancer remains a major challenge to public health. The estimated new cases and deaths from cancer in the United States in 2013 were 1,660,290 and 583,350, respectively (American Cancer Society, Cancer Facts & Figures. 2013: Atlanta: American Cancer Society (2013), which is hereby incorporated by reference in its entirety). Conventional cancer chemotherapy has been largely unable to meet the challenge posed by the great complexity of cancer cells (Hanahan et al., “The hallmarks of cancer,” Cell 100:57 (2000); Hanahan et al., “Hallmarks of cancer: The next generation,” Cell 144:646 (2011); Doroshow, “Overcoming resistance to targeted anticancer drugs,” N. Engl. J. Med. 369:1852 (2013), which are hereby incorporated by reference in their entirety) that causes cancer drug resistance (Hanahan et al., “Hallmarks of cancer: The next generation,” Cell 144:646 (2011); Holohan et al., “Cancer drug resistance: An evolving paradigm,” Nat. Rev. Cancer 13:714 (2013), which are hereby incorporated by reference in their entirety) and metastasis (Hanahan et al., “Hallmarks of cancer: The next generation,” Cell 144:646 (2011); Gupta et al., “Cancer metastasis: Building a framework,” Cell 127:679 (2006), each of which is hereby incorporated by reference in its entirety). The present invention affords an innovative approach that differs from the conventional ones for overcoming cancer drug resistance.

For forming a nanofibril network internally of cells, including cancer cells, the method involves contacting a cell that internally expresses a hydrolytic enzyme (an endoenzyme) with the peptide of the present invention or the pharmaceutical composition of the present invention, where the contacting is effective to cause cell uptake of the peptide followed by enzymatic cleavage of the peptide from the taurine/hypotaurine residue and then in situ self-assembly of the peptide to form a nanofibril network internally of the cell. As a consequence of forming the nanofibril network internally of the cell surface, one or more of the following occurs: cell migration is inhibited, cell survival is inhibited, and/or cell growth and division is inhibited. Overall, cellular processes are disrupted, and cell viability is reduced. Where a cytotoxic drug is conjugated to the peptide, drug release from the peptide allows for enhanced cytotoxicity. The cell can be ex vivo or in vivo (in accordance with the method of treatment described below).

Treatment of a patient for cancer involves administering to a subject having a cancerous condition a therapeutically effective amount of the peptide of the present invention or the pharmaceutical composition of the present invention, wherein the administering is effective to cause cell uptake of the peptide followed by enzymatic cleavage of the peptide from taurine/hypotaurine (predominantly in cancer cells), and then in vivo self-assembly of the peptides to form a nanofibril network within the cancer cells expressing an endoenzyme having enzymatic activity suitable to cleave the enzymatically-cleavable moiety. Such self-assembly has the effects noted above, and in the presence of a conjugated cytotoxic agent intracellular release of that cytotoxic agent is also afforded. Exemplary subjects include any mammal that is susceptible to cancerous conditions including, without limitation, rodents, rabbits, canines, felines, ruminants, and primates such as monkeys, apes, and humans.

Administration of the peptide or pharmaceutical composition can be carried out using any suitable approach. By way of example, administration can be carried out parenterally, subcutaneously, intravenously, intradermally, intramuscularly, intraperitoneally, by implantation, by intracavitary or intravesical instillation, intraarterially, intralesionally, intradermally, peritumorally, intratumorally, or by introduction into one or more lymph nodes. In certain embodiments, administration is carried out intralesionally, intratumorally, intradermally, or peritumorally.

In these several aspects of the invention, the cancer cells express an endoenzyme. In these embodiments, the enzyme produced by the cancer cells is an endoenzyme having hydrolytic activity, i.e., the enzyme hydrolyzes an ester group, carbonate group, thiocarbonate group, carbamate group, carboxylate group, or diacyl anhydride group that is present within the enzymatically cleavable moiety. The effect of such cleavage is liberation of the (hypo)taurine residue, which then affords hydrogelation internally of the cancer cells expressing the endoenzyme.

The cancer cells to be treated in accordance with these aspects can be present in a solid tumor, present as a metastatic cell, or present in a heterogenous population of cells that includes both cancerous and noncancerous cells. Exemplary cancer conditions include, without limitation, cancers or neoplastic disorders of the brain and CNS (glioma, malignant glioma, glioblastoma, astrocytoma, multiforme astrocytic gliomas, medulloblastoma, craniopharyngioma, ependymoma, pinealoma, hemangioblastoma, acoustic neuroma, oligodendroglioma, meningioma), pituitary gland, breast (Infiltrating, Pre-invasive, inflammatory cancers, Paget's Disease, Metastatic and Recurrent Breast Cancer), blood (Hodgkin's Disease, Leukemia, Multiple Myeloma, Lymphoma), lymph node cancer, lung (Adenocarcinoma, Oat Cell, Non-small Cell, Small Cell, Squamous Cell, Mesothelioma), skin (melanoma, basal cell, squamous cell, Kapsosi's Sarcoma), bone cancer (Ewing's Sarcoma, Osteosarcoma, Chondrosarcoma), head and neck (laryngeal, pharyngeal, and esophageal cancers), oral (jaw, salivary gland, throat, thyroid, tongue, and tonsil cancers), eye, gynecological (Cervical, Endrometrial, Fallopian, Ovarian, Uterine, Vaginal, and Vulvar), genitourinary (Adrenal, bladder, kidney, penile, prostate, testicular, and urinary cancers), and gastrointestinal (appendix, bile duct (extrahepatic bile duct), colon, gallbladder, gastric, intestinal, liver, pancreatic, rectal, and stomach cancers).

Use of the peptides and pharmaceutical compositions can be coordinated with previously known therapies. For instance, where the peptide is conjugated with a thermoablative nanoparticle, after formation of the pericellular nanofibril network, a tumor-containing region of the subject's body can be exposed to near infrared light, thereby causing thermal heating of the thermoablative nanoparticle and destruction of cancer cells covered by the nanofibril network. Alternatively, the peptides can be co-administered with cytotoxic or immunotherapeutic agents that are well known in the art.

In addition, chemotherapeutic agents, immunotherapeutic agents, or radiotherapeutic agents, as well as surgical intervention can be used in a coordinated manner with the peptides or pharmaceutical compositions of the present invention. Thus, a chemotherapeutic agent, an immunotherapeutic agent, or a radiotherapeutic agent can be administered to a patient before or after treatment with the peptides or pharmaceutical compositions of the present invention. Alternatively, surgical resection of a tumor can be carried out before or after treatment with the peptides or pharmaceutical compositions of the present invention. Optimization of such concurrent therapies is contemplated.

The accompanying Examples demonstrate the efficacy of a peptide of the invention with a platinum-containing cytotoxic drug (cisplatin), where even an otherwise non-cytotoxic dosage of the peptide affords a synergistic increase in the efficacy of cisplatin therapy. These same synergistic results should be obtained with other platinum-containing cytotoxic agents. Further, it is also contemplated that either additive or synergistic results can be achieved with other therapeutic agents of the type described herein.

### EXAMPLES

The following examples are intended to illustrate the present invention, but are not intended to limit the scope of the appended claims

### Materials and Methods for Examples 1-5

All the solvents and chemical reagents were used directly as received from the commercial sources without further purification. Esterase (from porcine liver) was purchased from Sigma-Aldrich.

Instruments:

LC-MS was performed on a Waters Acuity Ultra Performance LC with Waters MICRO-MASS detector. Hydrophilic products were purified with Waters Delta600 HPLC system equipped with an XTerra C18 RP column and an in-line diode array UV detector, hydrophobic products were purified with flash chromatography Hydrogen nuclear magnetic resonance (¹H-NMR) spectra were recorded on a Varian Unity Inova 400 with DMSO as solvent. Transmission electron microscope (TEM) images were taken on Morgagni 268 transmission electron microscope. Confocal images were taken on a Leica TCS SP2 Spectral Confocal Microscope. MTT assay for cell cytotoxicity was performed using a DTX880 Multimode Detector. Rheological data was obtained on TA ARES G2 rheometer with 25 mm cone plate.

TEM Sample Preparation:

Negative staining technique was used to study the TEM images. The 400 mesh copper grids coated with continuous thick carbon film (˜35 nm) were subjected to glowed discharge prior to their use to increase the hydrophilicity. After loading samples (4 μL) on the grid, the grids were rinsed with dd-water two or three times. Immediately after rinsing, the grid containing sample was stained with 2.0% w/v uranyl acetate three times. Afterwards, the grid was stained to dry in air.

General Procedures for Hydrogel Preparation:

Peptides 1-t/1 were dissolved into distilled water, and the pH of the solution was adjusted carefully by adding 1M NaOH, and this was monitored with pH paper. After the pH of the solution reached 7.4, extra distilled water was added to make the final concentration, followed by the addition of esterase to induce enzymatic gelation.

Cell Culture:

The HeLa cell line CCL-2™ was purchased from the American Type Culture Collection (ATCC, Manassas, Va., USA). The HeLa cells were propagated in Minimum Essential Media (MEM) supplemented with 10% fetal bovine serum (FBS) and antibiotics in a fully humidified incubator containing 5% CO₂ at 37° C.

Sample Preparation for Confocal Microscopy:

Cells in exponential growth phase were seeded in glass bottomed culture chamber at 1×10⁵ cell/well. The cells were allowed for attachment for 12 hours at 37° C., 5% CO₂. The culture medium was removed, and new culture medium containing 1-t, t or 2-t at 200 μM was added. After incubation for certain time, cells were stained with 1.0 μg/ml Hochst 33342 for 30 min at 37° C. in the dark. After that, cells were rinsed three times by PBS buffer, and then kept in the live cell imaging solution (Invitrogen Life Technologies A14291DJ) for imaging.

Cellular Uptake Measurement:

The following formula was used to calculate the intracellular concentration of the peptides.

Intracellular concentration=(c*300 uL)/(cell number*4*10⁻⁹ cm³)

where c=(fluorescence−15406.76945)/74174.36908. An average size for the common cells used in cell culture (HeLa) is 15-20 microns in diameter for a suspended cell (volume 4/3πr³=4000 μm³ or 4×10⁻⁹ cm³) (Moran et al., Cell 141:1262 (2010), which is hereby incorporated by reference in its entirety). Methanol was used to break the cell membrane and fully dissolve compounds 1-t, 2-t, and 3.

MTT Assay:

SKOV3 cells were seeded in exponential growth phase in a 96 well plate at a concentration of 1×10⁴ cell/well with 100 μL of McCoy's 5A medium modified supplemented with 10% fetal bovine serum (FBS), 100 U/mL penicillin and 100 μg/ml streptomycin. The cells were allowed to attach to the wells for 24 hours at 37° C., 5% CO₂. The culture medium was removed and 100 μL culture medium containing compounds (immediately diluted from fresh prepared stock solution of 10 mM) at gradient concentrations (0 μM as the control) was placed into each well. McCoy's 5A medium modified was regarded as blank. After culturing at 37° C., 5% CO₂ for 24 hours, 48 hours, and 72 hours, 10 μL of 5 mg/mL MTT (3-(4,5-Dimethylthiazol-2-yl)-2,5-diphenyltetrazolium bromide) was added to each well, and the plated cells were incubated in dark place for 4 hours. 100 μL 10% SDS with 0.01 M HCl was added to each well to stop the reduction reaction and to dissolve the purple formazan. After incubation of the cells at 37° C. for overnight, the OD at 595 nm of the solution was measured in a microplate reader.

WST Assay:

Cells in exponential growth phase were seeded in a 96 well plate at a concentration of 1×10⁴ cell/well. The cells were allowed to attach to the wells for 24 hours at 37° C. The culture medium was removed and 100 μL culture medium containing compounds (immediately diluted from fresh prepared stock solution of 10 mM) at gradient concentrations (0 μM as the control) was placed into each well. After the incubation of 72 hours, 10 μL of Cell Proliferation Reagent WST-1 was then added to each well and incubated for 2 hours at 37° C., 5% CO₂. The plate was shaken thoroughly for 1 min on a shaker to ensure homogeneous distribution of color. Subsequently, absorbance was measured at 450 nm in a microplate reader from which data points were collected.

### Example 1—Synthesis and Characterization of Hydrogelator Precursors

Hydrogelator precursors (1-t, 1) and control 2-t were prepared by combining solid phase and liquid phase peptide synthesis in fair yields (50-70%) and reasonable scales (0.1-0.5 g). The standard solid-phase peptide synthesis (SPPS) (Chan and White, Eds., Fmoc Solid Phase Peptide Synthesis: A Practical Approach, Oxford Univ Press (2000), which is hereby incorporated by reference in its entirety) uses 2-chlorotriyl chloride resin (100-200 mesh and 0.3-0.8 mmol/g) and N-Fmoc-protected amino acids with side chains properly protected. Before that, NBD-COOH was prepared from NBD-Cl based on literature (Cai et al., Anal. Chem. 86:2193 (2014), which is hereby incorporated by reference in its entirety), and then used directly in SPPS. The schemes shown in FIGS. 1A-B illustrate the synthetic procedures for making peptides 1-t/1, and the control peptide 2-t, respectively.

1-t: ¹H-NMR (400 MHz, DMSO-d₆) δ 9.27 (s, 1H), 8.20-8.11 (m, 3H), 7.94 (d, J=8.0 Hz, 1H), 7.77 (t, J=4.0 Hz, 1H), 7.68 (d, J=4.0 Hz, 1H), 7.50 (t, J=8.0 Hz, 1H), 7.37 (t, J=8.0 Hz, 1H), 7.21-7.05 (m), 4.44 (m, 1H), 3.90 (t, J=4.0 Hz, 1H), 3.61-3.50 (m, 4H), 3.27-3.17 (m, 2H), 3.10-3.07 (m, 4H), 2.92-2.85 (m, 1H), 2.81-2.72 (m, 1H), 2.64 (t, J=12.0 Hz, 1H), 2.57 (t, J=8.0 Hz, 1H), 2.46-2.43 (m, 3H), 2.29 (t, J=8.0 Hz, 1H). ESI MS (m/z) [M]+ calcd. for C₃₅H₄₀N₈O₁₂S, 797.3146 found [M-H]⁻ 795.2994.

1: ¹H-NMR (400 MHz, DMSO-d₆) δ 12.21 (s, 1H), 8.28 (d, J=8.0 Hz, 1H), 8.18 (d, J=8.0 Hz, 1H), 8.05 (t, J=4.0 Hz, 1H), 7.85 (d, J=8.0 Hz, 1H), 7.78 (d, J=8.0 Hz, 1H), 7.75 (d, J=8.0 Hz, 1H), 7.60 (s, 1H), 7.47 (m, 2H), 7.16-7.2 (m, 5-6H), 4.54 (td, J=8.0, 4.0 Hz, 1H), 4.47 (td, J=8.0, 4.0 Hz, 1H), 3.94 (t, J=8.0 Hz, 2H), 3.54 (q, J=12.0 Hz, 2H), 2.93-2.99 (m, 2H), 2.85-2.71 (m, 2H), ESI MS (m/z) [M]+ calcd. for C₃₃H₃₅N₇O₁₀, 689.3212 found [M-H]⁻ 687.2033.

2-t: ¹H-NMR (400 MHz, DMSO-d₆) δ 9.28 (s, 1H), 8.30-8.05 (m, 3H), 7.94 (d, J=8.0 Hz, 1H), 7.87 (t, J=4.0 Hz, 1H), 7.64 (d, J=16.0 Hz, 1H), 7.48 (t, 1H), 7.39 (t, J=8.0 Hz, 1H), 7.11-7.90 (m), 4.37-4.52 (m, 1H), 3.56 (m, 4H), 3.09 (m, 4H), 2.92-2.85 (m, 1H), 2.81-2.72 (m, 1H), 2.64 (t, J=12.0 Hz, 1H), 2.57 (t, J=8.0 Hz, 1H), 2.46-2.43 (m, 3H), 2.29 (t, J=8.0 Hz, 1H). ESI MS (m/z) [M]+ calcd. for C₃₅H₄₁N₉O₁₁S, 796.2745 found [M-H]⁻ 794.3245.

### Example 2—Cellular Uptake of D-peptide Conjugated to Taurine

It was unexpectedly observed that taurine, a non-proteingenic but essential amino acid (Huxtable, R. J., Physiol. Rev. 72:101-163 (1992); Price et al., J. Am. Chem. Soc. 125:13008-13009 (2003); Proshlyakov et al., J. Am. Chem. Soc. 126:1022-1023 (2004); Riggs-Gelasco et al., J. Am. Chem. Soc. 126:8108-8109 (2004), which are hereby incorporated by reference in their entirety) apparently enhanced cellular uptake of D-peptides. Conjugation of taurine and D-peptide derivative significantly boosted cellular uptake of the D-peptidic derivative, from below 100 μM (without the conjugation of taurine) to over mM inside cells, which is a more than 10-fold enhancement. Enzyme-catalyzed cleavage of the taurine group results in the self-assembly of the D-peptide derivatives and further enhances the intracellular accumulation of the D-peptide derivative (FIG. 2A). As the first example of taurine promoted cellular uptake, this work illustrates a fundamentally new strategy to deliver D-peptides into live cells.

As shown in FIG. 2B, molecule 1-t consists of a fluorophore (i.e., 4-nitro-2,1,3-benzoxadiazole (NBD)), a dipeptide residue (i.e., D-Phe-D-Phe), an enzyme (i.e., esterase) cleavage site (i.e., an ester bond), and a taurine residue. The NBD motif, exhibiting enhanced fluorescence in a hydrophobic environment, can efficiently indicate nanofibrils formed by molecular self-assembly in cells (Gao et al., Nat. Commun. 3:1033 (2012), which is hereby incorporated by reference in its entirety). The D-diphenylalanine peptide, besides serving as the self-assembly motif (Li et al., J. Am. Chem. Soc. 135:542-545 (2013); Shi et al., Biomacromolecules 15(10):3559-3568 (2014); Li et al., J. Am. Chem. Soc. 135:9907-9914 (2013); Reches et al., Science 300:625-627 (2003), which are hereby incorporated by reference in their entirety), ensures the proteolytic resistance of the molecules. The ester bond (between ethanolamine and succinic acid) allows esterase to catalyze the conversion of 1-t to 3. To verify the roles of taurine and the ester bond, two control molecules, 1 and 2-t were designed. Comparing to 1-t, 1 lacks the taurine residue, and an amide bond in 2-t replaces the ester bond in 1-t. Based on the design in FIG. 2B, solid phase and liquid phase peptide synthesis (FIGS. 1A-B) were combine to produce these molecules in fair yields (50˜70%) and reasonable scales (0.1-0.5 g) after the purification of the crude product by flash chromatography and a reverse phase HPLC.

Benefiting from advances in supramolecular chemistry, the fundamental understanding of the formation of molecular nanofibrils or the corresponding hydrogels has improved considerably in the past decade (Lu et al., J. Am. Chem. Soc. 125:6391-6393 (2003); Lakdawala et al., J. Am. Chem. Soc. 124:15150-15151 (2002); Nuraje et al., J. Am. Chem. Soc. 126:8088-8089 (2004); Gao et al., Adv. Mater. 17:2037-2050 (2005); Boekhoven et al., J. Am. Chem. Soc. 134:12908-12911 (2012); Minkenberg et al., Chem. Commun. 48:9837-9839 (2012), which are hereby incorporated by reference in their entirety). To elucidate the enzyme-catalyzed self-assembly to form supramolecular nanofibrils inside cells after cellular uptake illustrated in FIG. 2A, the enzymatic transformation of 1-t (or 1) to 3 and the subsequent self-assembly of 3 at the concentration of 1.0 wt % was first examined. To study the enzymatic hydrogelation process of 1-t, analytical HPLC was used to evaluate the rate of enzymatic transformation and transmission electron microscopy (TEM) to examine the self-assembly. At pH=7.4, 1-t formed a transparent orange solution (FIG. 6A), indicating that the incorporation of taurine significantly enhanced the solubility of 1-t. After being treated with esterase (1 U/ml) for 1 min, the orange solution remained unchanged, though analytical HPLC showed that the esterase already converted 2.5% of 1-t to 3 (1-t:3=30:1). At this moment, TEM of the solution revealed that there were short nanofibrils with uniform diameters (8±2 nm) and a few long nanofibrils with diameters of 12±2 nm (FIG. 6A). After 24 hours, the solution of 1-t turned into a hydrogel, with about 11% of 1-t (1-t:3=8:1) being converted to 3 according to HPLC. TEM of the hydrogel showed that the self-assembled nanofibrils (d=8±2 nm) entangle with each other to form the matrices of the hydrogel (FIG. 6B), within which are sporadic short nanofibrils with diameters of 12±2 nm (FIG. 6B). These short nanofibrils were of the same density as appearing in the solution shown in FIG. 6A. The above results indicated that i) 1-t exists mainly as monomer or small oligomer (which cannot be detected by TEM and is readily available to esterase), but has weak tendency to form nanofibrils in solution even at a high concentration; ii) 3 and 1-t are able to co-self-assemble to form nanofibrils. These features allow the enzymatic transformation of 1-t to 3 to catalyze the formation of the nanofibrils and the subsequent hydrogelation. The control molecule 1, without the taurine residue, was less soluble than 1-t and only formed semitransparent dispersion. After the treatment of esterase (1 U/ml), the resulted hydrogel formed by the hydrolysis of 1 was rather soft and exhibited yellow fluorescence (FIGS. 7A-B).

Although the hydrogelation test is the most convenient way to identify molecular self-assembly in solution, the self-assembly or aggregation is also able to occur at a lower concentration than that needed for the formation of a hydrogel. To better elucidate the self-assembly of those dipeptide derivatives for cell experiment, the self-assembly of 1-t at a lower concentration (i.e., 100 μM) than that of the critical concentration of hydrogelation (1.0 wt %, 12.6 mM) was evaluated.

As shown in the TEM (FIG. 3A), there was hardly any aggregate in the solution of 1-t at the concentration of 100 μM. After the addition of esterase (1 U/mL) to the solution for 24 hours, large amount of nanofibrils (d=8±2 nm) appeared, interwoven with some aggregates (FIG. 3B). Correspondingly, the static light scattering (SLS) of the solution before esterase treatment exhibited low signal (almost the same intensity as that of PBS buffer), but the SLS signal increased dramatically after the addition of esterase (1 U/mL) (FIG. 3C), suggesting that the conversion of 1-t to 3 at 100 μM results in the self-assembly of 3. HPLC revealed that more than 50% of 1-t has been converted to 3, suggesting that 1-t and 3 can co-self-assemble to form the nanofibrils. This result is particularly noteworthy because it confirmed that enzymatic conversion can rapidly and easily trigger the formation of nanofibrils. In addition to TEM and light scattering, the fluorescence associated with NBD may serve as an efficient assay to report the self-assembly (Gao et al., Nat. Commun. 3:1033 (2012), which is hereby incorporated by reference in its entirety), which fluoresce with increased quantum yield because the self-assembled nanofibrils or aggregates encapsulate the NBD residues in highly hydrophobic environment (Soltani et al., J. Biol. Chem. 282:15709-15716 (2007), which is hereby incorporated by reference in its entirety). As shown in FIG. 3D, significant fluorescence appeared in the solution treated with 1 U/mL esterase, compared with that of the solution prior to the addition of enzyme (inset in FIG. 3D).

The fluorescence contrast before and after the formation of nanofibrils allowed the evaluation of cellular uptake (as well as intracellular self-assembly, the dynamics, the localization of the nanofibrils) of the small molecules in live cells by confocal microscopy. Thus, HeLa cells were incubated with 1-t and 1, respectively, at the concentration of 200 μM in culture medium, 37° C., for 24 hours and then the confocal microscopy images were taken to examine the cellular distribution of these molecules (1-t (or 1) and 3). Here, 200 μM was chosen for a shorter experiment period, better imaging quality, and easier comparison with the control. These results showed that HeLa cells treated with 1-t exhibited significant fluorescence in cytoplasm (FIG. 4), indicating that the cells uptake large amount of 1-t, some of which turned into 3 upon esterase catalyzed hydrolysis, and the molecules of 3 co-self-assembled with 1-t to form intracellular nanofibrils that exhibited strong fluorescence. In contrast, confocal microscopy images of the HeLa cells treated by 1 hardly showed any yellow fluorescence inside cells (FIG. 4). Moreover, the background fluorescence outside the cells was brighter than that of the cells treated by 1, indicating that little amount of 1 enters the cells. These results confirmed that the incorporation of taurine boosted cellular uptake of the D-peptide derivatives. To prove that the cleavage of the ester bond is critical for the significant fluorescence observed in the cytosol for the cells treated by 1-t, the cellular uptake of 2-t as a control was examined since the amide bond in 2-t hydrolyzes slower than the ester bond in 1-t does. There were a few yellow bright spots inside cells after 24-hour incubation of 2-t, likely due to endosomes containing 2-t (FIG. 8). This result proved that self-assembly due to ester bond hydrolysis facilitated the intracellular accumulation of the D-peptide derivatives in the cytosols. The above results, thus, confirmed that the conjugation of taurine drastically facilitated the cellular uptake of the D-peptide derivatives.

To quantify the intracellular concentration of the D-peptide derivatives, the procedure illustrated in FIG. 9A was used to determine the cellular uptake of 1-t, 1, and 2-t after treating HeLa cells with these molecules, respectively. As shown in FIG. 5A, when being incubated with 200 μM of 1-t for 24 hours, HeLa cells uptake 1-t to reach an uptake concentration of 1.6 mM, 8 times of the extracellular concentration (200 μM) of 1-t in the culture medium. The “uptake concentration (Cᵤ)” of 1-t or 1 is the sum of 3 and 1-t (or 1) inside the cells. On the contrary, the presence of 1 (or 3) inside the HeLa cells was hardly detected when incubating with 200 μM of 1. This striking contrast, again, verified that the covalent incorporation of taurine residue into the D-peptide derivatives significantly increased the cellular uptake. Besides, the intracellular concentration of 2-t in HeLa cells incubated with 200 μM of 2-t for 24 hours is 0.8 mM, half of the Cᵤ of 1-t in cells treated with 1-t (200 μM) for 24 hours, suggesting that intracellular esterase catalyzed co-self-assembly of the D-peptide derivatives promotes the cellular accumulation based on the taurine-enhanced uptake. The Cᵤ of 2-t (800 μM) is 4 times as concentrated as incubating concentration in culture medium (200 μM), indicating a higher rate of uptake than diffusion or efflux. It was found that the cellular uptake of 1-t gradually rose with increasing incubation time. Notably, the Cᵤ of 1-t kept going up even after 48-hour incubation with 1-t, confirming that the formation of nanofibrils by the enzyme-catalyzed self-assembly maintained the cellular uptake of molecule 1-t (FIG. 5B). According to the time-dependent intracellular concentration curve (FIG. 9B), the cellular uptake of 1-t started to slow down after 10 hours, probably due to the finite amount of esterase inside cells, and the Cᵤ of 1-t reaches 2.4 mM at 48 hours.

HeLa cells were also incubated with 1-t at different concentrations (i.e., 25 μM, 50 μM, 100 μM, 200 μM, and 500 μM), and then the corresponding cellular uptake after 24 hours was examined. As shown in FIG. 5C, when the incubation concentration is below or at 25 μM, the enhancement of cellular uptake was less pronounced (e.g., the intracellular concentration is 28 μM when the incubation medium contains 25 μM of 1-t). According to FIG. 5C, 200 μM appeared to be the optimum incubation concentration among the tested ones of 1-t, which reaches an 8-fold enhancement of cellular uptake (compared with the incubation concentration) after 24 hours of incubation (and 12-fold after 48 hours). While the incubation concentrations are 50 μM and 500 μM, the boosts were about 3-fold and 4.5-fold, respectively, at 24 hours. These results indicate that the activity of enzymes and the minimum aggregation concentration together dictate the enhancement of the cellular uptake. In addition, cell extraction analysis (of the HeLa cells incubated with 1-t or 2-t at the concentration of 200 μM for 24 hours) by analytical HPLC showed that the intracellular ratio of 3 and 1-t is about 1:4 while 2-t remained intact inside cells (FIG. 10), further confirming the enzymatic transformation of 1-t to 3 and the proteolytic resistance of 1-t, 2-t, and 3.

To evaluate the retention of the D-peptide derivatives inside cells, the HeLa cells were washed with fresh PBS buffer after 24 hour incubation of the cells with 200 μM of 1-t or 2-t and the intracellular concentration of D-peptide derivatives was measured (the Cᵤ of 1-t or the concentration of 2-t), respectively. As show in FIG. 5D, each wash only slightly decreased the intracellular concentration of D-peptide derivatives (3 and 1-t). After washing five times with PBS buffer, Cᵤ of 1-t (the total intracellular concentration of 3 and 1-t) almost remained the same (from 1.6 mM to 1.4 mM). On the contrary, each wash decreased the intracellular concentration of 2-t by about 25% (e.g., from 0.8 mM to 0.6 mM after first wash). After washing the cells five times with PBS buffer, the intracellular concentration of 2-t dropped dramatically (i.e., about an order of magnitude, from 0.8 mM to 0.08 mM). These results confirm that the intracellular cleavage of taurine and the formation of the nanofibrils composed of 3 ant 1-t significantly reduce the diffusion of 3 and 1-t outside of the cells.

### Example 3—Synthesis and Characterization of Hydrogelator Precursors

The synthesis of D-10/L-10 and D-12/L-12 was simple and straightforward, using essentially the same synthetic strategy of Example 1 except that 2-napthaleneacetic acid was used to form a 2-napthylacetyl group at the N-terminus of the peptide. The facile synthetic route (FIG. 11) combined liquid-phase synthesis and solid-phase peptide synthesis (SPPS) for making the precursors. For example, by loading N-Fmoc-protected phenylalanine (Fmoc-Phe-OH) onto 2-chlorotrityl resin and carrying out SPPS, Nap-FF was obtained (Zhang et al., Langmuir 27:529-537 (2011), which is hereby incorporated by reference in its entirety) for coupling with ethanolamine to produce L-12. After L-12 reacted with succinic anhydride, forming L-7, another step of amide bond formation allowed the attachment of taurine to L-7 thereby forming L-10. After the purification by HPLC, the overall yield of L-10 was about 60%.

The same synthetic approach, albeit using Fmoc-Phe_{D}-OH, produced D-10 (as well as D-7 and D-12). Control molecule D-11 was prepared by directly reacting taurine with Nap-ff (using step J conditions, FIG. 11) in place of coupling with ethanolamine and then succinic anhydride (i.e., steps H and I, FIG. 11).

¹H-NMR confirmed that the intended products were recovered.

### Example 4—In Vitro Inhibition of Cancer Cell Survival by D-Peptide Conjugated to Taurine

D-7 and D-10 (FIG. 12A), the enantiomers of L-7 and L-10, were prepared as described in Example 3. HeLa and SKOV3 cells were used in an MTT assay for assessing cytotoxicity of the peptides against these cancer cell lines.

D-7 exhibited higher activity against HeLa cells than L-7 (FIG. 12B). The cell uptake of the D-peptide precursor was further increased by conjugating taurine to D-7 to form D-10 (FIG. 12A), which exhibited drastically increased activity against HeLa cells (FIG. 12B). Most importantly, there was nearly an order of magnitude increase from the activity of L-7 to that of D-10, indicating that intracellular nanofibers of D-peptides were a promising candidate for inhibiting cancer cells. In fact, D-10 (50 μM) inhibited over 70% of SKOV3 ovarian cancer cells in vitro (FIG. 12B), an activity that is comparable to the IC₅₀ of cisplatin (35 μM) (Oumzil et al., “Nucleolipids as Building Blocks for the Synthesis of 99mtc-labeled Nanoparticles Functionalized with Folic Acid,” New J Chem 38:5240 (2014), which is hereby incorporated by reference in its entirety). In addition, D-11, an analogue of D-10 that lacked the ester bond and was unable to self-assemble inside cells, was completely cell compatible (FIG. 12B). These results validate that esterase-instructed intracellular nanofibers of D-peptides promise a new paradigm for inhibiting drug resistant cancer cells.

Because the nanofibers would dissociate into monomers after the death of cancer cells, the self-assembling building block is unlikely to cause chronic toxicity. This unique advantage makes the molecular nanofibers attractive candidates for combination therapy.

### Example 5—Enzyme-Instructed Intracellular Molecular Self-Assembly to Boost Activity of Cisplatin Against Drug-Resistant Ovarian Cancer Cells

Since its serendipitous discovery five decades ago (Rosenberg et al., Nature 205:698-699 (1965), which is hereby incorporated by reference in its entirety), cisplatin has become one of the most successful therapeutic agents for anticancer chemotherapy (Rosenberg et al., Nature 222:385-386 (1969); Rosenberg, B., Cancer 55:2303-2316 (1985), which are hereby incorporated by reference in their entirety). Particularly, cisplatin has drastically extended the progression-free survival (PFS) of patients with ovarian cancers (Armstrong et al., Gynecologic Oncology Grp., New Engl. J Med. 354:34-43 (2006), which is hereby incorporated by reference in its entirety). However, owing to the lack of early detection of ovarian cancer and the almost inevitable relapse in the patients with advanced ovarian cancer, drug resistance remains a major obstacle in treating ovarian cancers (Yap et al., Nat. Rev. Cancer 9:167-181 (2009); Parkin, D. M., Lancet Oncol. 2:533-543 (2001); Greenlee et al., CA-Cancer J. Clin. 51:15-36 (2001); Rabik et al., Cancer Treat. Rev. 33:9-23 (2007); Siegel et al., Ca-cancer J. Clin. 65:5-29 (2015), which are hereby incorporated by reference in their entirety). Many approaches have been investigated to address the urgent need of treating drug-resistant ovarian cancers. One of the most explored strategies is combination chemotherapy, such as the combination of cisplatin with other therapeutics, because the advantages of cisplatin promote the rapid translation from preclinical to clinical settings. Despite the remarkable clinical success of combination therapies (Armstrong et al., Gynecologic Oncology Grp., New Engl. J. Med. 354:34-43 (2006), which is hereby incorporated by reference in its entirety), the 5-year relative survival rate of ovarian cancer hardly improved over the past decade (45% between 2004-2010 vs. 45% between 1996-2003) (Siegel et al., Ca-cancer J. Clin. 65:5-29 (2015), which is hereby incorporated by reference in its entirety). Thus, there remains an urgent need for innovative approaches in cisplatin-based combination therapies.

While Examples 1-4 demonstrate enzyme-instructed intracellular molecular self-assembly and inhibition of cancer cell survival, this Example focuses on the use of D-peptides for intracellular enzyme-instructed self-assembly in combination with cisplatin treatment. Two enantiomeric peptidic precursors (L-10 and D-10) that turn into the self-assembling molecules (L-12 and D-12) upon the catalysis of carboxylesterases (CES; FIG. 13) were designed and synthesized as described in Example 3.

After obtaining the precursors, the use of CES to convert the precursors into the hydrogelators that self-assemble in water to form molecular nanofibers was tested. The addition of L-10 or D-10 in PBS buffer at pH 7.4 at a concentration of 0.4 wt % (5.5 mM) afforded a transparent solution. After the addition of CES (2 U/mL) into the solution of L-10 or D-10, a translucent hydrogel formed after 24 hours. The minimum gelation concentration (mgc) of L-12 or D-12 was found to be about 0.1 wt % (1.4 mM). While CES efficiently converted both L-10 and D-10 into L-12 and D-12, respectively, the hydrogel of L-12 was apparently weaker than the hydrogel of D-12. Without being bound by belief, it is believed that this subtle difference might originate from weaker interactions between D-12 and CES than between L-12 and CES. The transmission electron microscopy (TEM) images of the resulting hydrogels revealed the formation of uniform nanofibers after the addition of CES (FIGS. 14A-B). The diameters of the nanofibers of the hydrogel formed by L-12 or D-12 after the addition of CES in the solution of L-10 or D-10 were 10±2 nm or 8±2 nm, respectively (FIGS. 14A-B).

Example 4 illustrated the cytotoxicity of L-10 and D-10, indicating that L-10 and D-10 showed significant cytotoxicity to SKOV3 cells at concentrations below the mgc. Thus, static light scattering (SLS) was used to help verify the existence of nanoscale assemblies (for example, nanofibers or nanoparticles) in the solution of L-10 or D-10 at concentrations lower than the mgc and after the addition of CES (2 U/mL). The concentrations from 10 μM to 100 μM were chosen to analyze whether there are differences in self-assembly of the molecules before and after the addition of CES. Before being treated with CES, the signal intensity ratios of the solution of L-10 or D-10 at concentrations from 10 μM to 50 μM were close to zero (FIGS. 14C-D), indicating that there are hardly any assemblies of L-10 or D-10 in the solution. When the concentration of the solution of L-10 or D-10 increased to 100 there was a slight increase of intensity ratio, indicating that small amounts assemblies of L-10 or D-10 exist in the solution. In contrast, the addition of CES to the solution of L-10 or D-10 at concentrations from 10 μM to 100 μM resulted in a significant increase of the signal intensity ratios, especially when the concentration of L-10 or D-10 was at or above 50 μM. For example, the signal intensity ratio of the solution of L-10 or D-10 at 50 μM drastically increased from about zero (before the addition of CES) to about 17 (after the addition of CES), which revealed the formation of assemblies of L-12 or D-12, respectively. Moreover, the solution of 100 μM L-10 showed a 9-fold increase of the signal intensity ratio after the addition of CES, indicating the formation of a larger amount of assemblies after enzymatically converting the precursors to the hydrogelators. Similarly, the signal intensity ratio of the solution of 100 μM D-10 increased significantly after the addition of CES, which agrees with the observation that CES converts D-10 into D-12 to form self-assembling nanoscale assemblies in water (FIG. 14B).

After confirming that CES converts the precursor L-10 or D-10 into the hydrogelator L-12 or D-12, the stability of the precursors (L-10 or D-10) when incubated with the ovarian cancer cells was determined. After culturing the precursors with SKOV3 or A2780cis cells at 37° C. for 4 hours, the cell lysates and culture medium were collected for liquid chromatography-mass spectrometry (LC-MS) analysis and the intracellular concentrations of the precursors, the hydrogelators, and the relevant proteolyzed products were determined. After incubation with SKOV3 or A2780cis cells for 4 hours, more than 85% of the precursors (L-10 or D-10) turned into the corresponding hydrogelators (L-12 or D-12; see Table 1 below).

TABLE 1
The intracellular concentrations of the precursors and hydrogelators in
SKOV3 and A2780cis cells

| Compound | Precursor (10) [μm] | Hydrogelator (12) [μm] | Ratio^{[a]} |
|---|---|---|---|
| L-10^{[b]} | 62 | 431 | 6.95 |
| D-10^{[b]} | 16 | 108 | 6.75 |
| D-10^{[c]} | 69 | 582 | 8.43 |

^{[a]}Ratio of hydrogelator to precursor after 4 hours.
^{[b]}The cell lysates of SKOV3 cells were collected after 4 hours incubation with 20 μm (15 μg/mL) of D-10 or with 50 μm (37 μg/mL) of L-10 at 37° C.
^{[c]}The cell lysates of A2780cis cells were collected after 4 hours incubation with 100 μm (73 μg/mL) of D-10 at 37° C.

Moreover, the intracellular concentrations of the hydrogelators were all above 100 μM, which indicated the intracellular self-assembly of the hydrogelators. The cumulative intracellular concentration of L-10 and L-12 was also about 10-fold higher than the incubation concentration of L-10, and the cumulative intracellular concentration of D-10 and D-12 was about 5-fold higher than the incubation concentration of D-10. These results not only indicated that the cellular uptake of L-10 is more efficient than that of D-10, but also confirmed that the selective retention of hydrogelators inside the cells originates from ester bond cleavage catalyzed by CES. A fluorescent esterase substrate, 6-CFDA (Riordan et al., Anticancer Res. 14:927-931 (1994), which is hereby incorporated by reference in its entirety), also confirmed high esterase activity in SKOV3 cells. The culture medium containing L-10 or D-10, which was incubated with SKOV3 cells or A2780cis cells was also analyzed. After 4 hours incubation with SKOV3 cells, about 19% of L-10 in the medium turned into L-12 (see Table 2 below), and the concentration of L-10 in the medium decreased from 50 μm to 39 μm; about 15% of D-10 converted D-12, and the concentration of D-10 in the medium decreased from 20 μm to 16 μm. A similar trend was also observed in A2780cis cells. These results further validated the belief that intracellular enzymatic conversion of the precursors catalyzed by CES results in the intracellular self-assembly of the hydrogelators.

TABLE 2
Summary of IC₅₀ and IC₉₀ values of the precursors against the ovarian
cancer cells for 48 hours

| | |
|---|---|
| SKOV3 Cells | A2780cis Cells |

| Compound | IC₅₀ | IC₉₀ | IC₅₀ | IC₉₀ |
|---|---|---|---|---|
| L-10 | 62 | 78 | 94 | 98 |
| D-10 | 48 | 53 | 69 | 73 |

To evaluate the effect of intracellular self-assembly of L-12 or D-12 for cisplatin-based combination therapy, the cell viability of three ovarian cancer cell lines was tested by incubating them with the mixture of precursors and cisplatin (CP). After 72 hours, the mixture of CP (6 μg/mL) with D-10 (15 μg/mL) or L-10 (37 μg/mL) inhibited about 74% or 87%, respectively, of SKOV3 cells (FIG. 15A). In contrast, D-10 (15 μg/mL) or L-10 (37 μg/mL) alone was almost innocuous to the cells, and CP (6 μg/mL) alone inhibited only 48% SKOV3 cells (FIG. 15A). Another method to treat the SKOV3 cells was also used, in which D-10 or L-10 was added 12 hours after the addition of CP to SKOV3 cells. As shown in FIG. 15A, 72 hours after the addition of D-10 (15 μg/mL) or L-10 (37 μg/mL) following the addition of CP (6 μg/mL), the inhibition of SKOV3 was about 80% or 86%, respectively. The higher efficacy exhibited by L-10 agreed with the higher uptake and incubation concentration of L-10.

The combination of CP and D-10 for treating A2780cis (cisplatin-resistant) and A2780 (cisplatin-sensitive) cells was also tested. D-10 (15 μg/mL) alone hardly exhibited any cytotoxicity to A2780cis cells (FIG. 15B). The combination of D-10 and CP inhibited 70% of A2780cis cells, which was double the activity of CP. The combination of D-10 and CP significantly inhibited A2780 cell viability and decreased the viability of A2780 from about 38% (without adding D-10) to only 9%. Since SKOV3 and A2780cis are two drug-resistant ovarian cell lines, CP showed lower inhibition ability against these two cell lines compared with A2780 cells. These results confirm that the addition of the precursors of self-assembling small molecules in combination with cisplatin drastically boosts the activity of cisplatin against drug-resistant ovarian cancer cells. The IC₅₀ values of L-10 and D-10 against the ovarian cancer cells were 62-94 μm and 48-69 μm, respectively (Table 2 above), but their concentrations for the combination therapy can be lower than IC₅₀ values because enzyme-induced assembly causes intracellular accumulation of the hydrogelators. Furthermore, the intracellularly formed nano-fibers (of D-10) were about seven times more effective against HeLa cells than the nanofibers of the dipeptides reported previously (Nap-FF (Kuang et al., Angew. Chem. Int. Ed. 52:6944-6948 (2013); Angew. Chem. 125:7082-7086 (2013), which is hereby incorporated by reference in its entirety); see Table 3 below.

TABLE 3
Summary of IC₉₀ values of the precursors and
NapFF against HeLa cells for 48 hours

| Compound | IC₉₀ |
|---|---|
| L-10 | 500 |
| D-10 | 78 |
| Nap-FF | 400 |

To verify the critical role of enzyme-instructed self-assembly, a control compound was synthesized, which replaces the ester bond in D-10 by an amide bond. This change (—COO— to —CONH—) rendered the control compound resistant to CES. Control compound (500 μm) alone hardly inhibited SKOV3 cells after 72 hours incubation. After 72 hours incubation with SKOV3 cells, while CP (6 μg/mL) alone caused about 40% cell death, the mixture of the control compound (15 μg/mL) and CP (6 μg/mL) inhibited only about 32% of SKOV3 cells. The innocuous effects of the control compound also excluded the possibility that L-10 or D-10 act as a surfactant to inhibit cell survival. A similar trend was observed in A2780cis cells. These results further confirmed that enzyme-instructed self-assembly inside cells was the main cause for boosting the efficacy of CP in the combination therapy of CP with the precursors (D-10 and L-10). Some of the cell viabilities slightly exceed 100% (for example, FIG. 15B), because the MTT assay measured the activity of mitochondrial reductase and it was not unusual for treated groups to have higher enzyme activity than the control group.

To gain insight into the action of intracellular self-assembly within cells, the change of the actin filaments inside cells was examined. SKOV3 cells treated by D-10 (20 μM (15 μg/mL), 20 hours) exhibited far fewer well-defined, long actin filaments than those in the control SKOV3 cells without the treatment of D-10 (FIG. 16). This trend became more pronounced after the concentration was increased from 20 to 100 μM, as evidenced by the number of the actin filaments in the cells (FIG. 17). This observation agreed with the belief that the intracellular nanofibers of small peptides interact with actin (Kuang et al., J. Biol. Chem. 289:29208-29218 (2014), which is hereby incorporated by reference in its entirety). To verify the reversible assembly of D-12 inside cells, the SKOV3 cells were treated with D-10 at the concentrations of 20, 50, and 100 μm respectively, for 20 hours, then the media was replaced with fresh media and the cells were incubated for an additional 20 hours. Actin filaments recovered after being treated with fresh medium for 20 hours when the concentrations of D-10 were 20 and 50 μM. The incomplete recovery of actin filaments, when [D-10]=100 μM, supports the belief that D-10 starts to self-assemble at 100 μM. After being incubated with L-10, SKOV3 cells exhibited similar behavior after 20 hours, cells incubated with L-10 (50 μM) exhibited fewer well-defined actin filaments compared with the cells without the treatment of L-10. However, 20 hours after exchanging the media, the morphology of actin filaments was restored to normal. These results indicate that intracellular nanofibers formed by enzyme-instructed self-assembly exhibit transient cytotoxicity that should help minimize long-term systemic burden in combination therapy. Dissociation likely reduces the long-term cytotoxicity after the apoptosis of cells so that the precursors and nanofibers cause minimal systemic toxicity.

In conclusion, it was demonstrated that enzyme-instructed intracellular self-assembly of small molecules is a new approach to boost the activity of CP against two drug-resistant ovarian cancer cell lines. Moreover, at the optimal concentrations, 20 μM (D-10) and 50 μM (L-10) used for boosting the activities of the cisplatin, L-10 and D-10 hardly inhibited HS-5 and PC-12 cells, despite cisplatin significantly inhibiting HS-5 and PC-12 cells (Mendonca et al., Mutat. Res. Genet. Toxicol. Environ. Mutag. 675:29-34 (2009), which is hereby incorporated by reference in its entirety). Moreover, intravenous injection of L-10 or D-10 at doses of 5 to 20 mg/kg hardly affected the weight and organ index of mice after 7 days (FIGS. 18A-C), confirming the low systemic toxicity of the precursors.

The genome analysis according to The Cancer Genome Atlas (TCGA) indicated the amplification of CES in certain tumors (for example, breast and ovarian cancer; FIG. 19), which not only supports the utility of the invention for treating various other cancers based on the self-assembly of intracellular nanofibers. This work, together with other emerging evidence (Yang et al., Acc. Chem. Res. 41:315-326 (2008); Gao et al., Langmuir 29:15191-15200 (2013); Gao et al., ACS Nano 7:9055-9063 (2013); Gao et al., Nat. Commun. 3:1033 (2012); Yang et al., Adv. Mater. 19:3152-3156 (2007); Kuang et al., J. Biol. Chem. 289:29208-29218 (2014); Kuang et al., Angew. Chem. Int. Ed. 52:6944-6948 (2013); Angew. Chem. 125:7082-7086 (2013); Tanaka et al., J. Am. Chem. Soc. 137:770-775 (2015); Pires et al., J. Am. Chem. Soc. 137:576-579 (2015); Zorn et al., J. Am. Chem. Soc. 133:19630-19633 (2011); Schneider et al., J. Am. Chem. Soc. 124:15030-15037 (2002); Newcomb et al., Nat. Commun. 5:3321 (2014), which are hereby incorporated by reference in their entirety), indicates that enzyme-instructed self-assembly promises a new way for developing combination therapy for cancer treatment. Because other platinum-containing drugs are available, such as carboplatin (Ozols et al., J. Clin. Oncol. 21:3194-3200 (2003), which is hereby incorporated by reference in its entirety), it is believed that the results obtained with cisplatin should extend to other platinum-containing drugs if not cytotoxic cancer drugs as whole class.

### Example 6—Selectively Inhibiting Cancer Cells by Intracellular Enzyme-Instructed Assembly Using L-10 and D-10

Reagents:

The reagents, such as N, N-diisopropylethylamine (DIPEA), O-benzotriazole-N,N,N′,N′-tetramethyluronium-hexafluorophosphate (HBTU), N,N′-diisopropylcarbodiimide (DIC), N-hydroxysuccinimide (NHS), and taurine were purchased from ACROS Organics USA.

Cell Culture:

All cell lines were purchased from the American Type Culture Collection (ATCC, Manassas, Va., USA). Cells were seeded in the density of 10⁴ cells/well in 96 well plates. U87MG, T98G, HepG2, HeLa, and MCF-7 cells were maintained in MEM medium supplemented with 10% fetal bovine serum (FBS), 100 U/mL penicillin, and 100 μg/ml streptomycin. A375 and HS-5 cells were maintained in DMEM supplemented with 10% fetal bovine serum (FBS), 100 U/mL penicillin, and 100 μg/ml streptomycin. MES-SA, MES-SA/D×5, and SKOV3 cells were maintained in McCoy's 5A medium modified supplemented with 10% fetal bovine serum (FBS), 100 U/mL penicillin, and 100 μg/ml streptomycin. A2780, A2780cis, and HCC1937 cells were maintained in PRMI-1640 medium supplemented with 10% fetal bovine serum (FBS), 100 U/mL penicillin, and 100 μg/ml streptomycin. Saos-2 cells were maintained in McCoy's 5A medium modified supplemented with 15% fetal bovine serum (FBS), 100 U/mL penicillin, and 100 μg/ml streptomycin. PC12 cells were maintained in F-12K supplemented with 15% horse serum, 2.5% fetal bovine serum (FBS), 100 U/mL penicillin, and 100 μg/ml streptomycin. All cells were grown at 37° C., 5% CO₂.

MTT Assay:

The cells were seeded in exponential growth phase in a 96 well plate at a concentration of 1×10⁴ cell/well with 100 μL of culture medium. Cells were allowed to attach for 24 hours at 37° C., 5% CO₂ and then the culture medium was removed with the help of vacuum pump. Culture medium containing the precursors in gradient concentrations was added to each well (0 μM as control). 10 μL of 5 mg/mL MTT ((3-(4,5-Dimethylthiazol-2-yl)-2,5-diphenyltetrazolium bromide) was added to each well after 24 hours, 48 hours, and 72 hours incubation. After 4 hours incubation of the plated cells in dark place, 100 μL 10% SDS with 0.01 M HCl was added to each well to dissolve the purple formazan and to stop the reduction reaction. 24 hours later, the absorbance of the solution at 595 nm was finally measured with a DTX 880 Multimode Detector.

The cytotoxicity of L-10 or D-10 on multiple cell lines including cancer cells and normal cells was tested (FIG. 20A-O). The IC₅₀ values of the third day (i.e., 72 h) in μg/mL are summarized in FIG. 21A-B. The precursors were tested on two breast cancer cell lines—HCC1937, a line of triple negative breast cancer cells (TNBC), and MCF-7, a common breast cancer cell line. As summarized in FIGS. 20A-B and FIGS. 21A-B, L-10 and D-10 were very effective against these two cell lines. The IC₅₀ values of L-10 for HCC1937 and MCF-7 were 29 and 28 μg/mL, respectively; the IC₅₀ values of D-10 for HCC1937 and MCF-7 were 26 and 25 μg/mL, respectively. Being incubated with a drug sensitive (A2780) and two drug resistant (A2780cis and SKOV3) ovarian cancer cell lines, L-10 gave the IC₅₀ values of 49, 39 and 46 μg/mL against A2780, A2780cis, and SKOV3, respectively (FIGS. 20C-E and 21A). Similar to the case of inhibiting breast cancer cells, D-10 exhibited higher inhibitory activity than L-10, with IC₅₀ values at 37, 36 and 31 μg/mL, respectively, against those three cell lines (FIGS. 20C-E and 21B). Notably, both precursors (i.e., L-10 and D-10) were slightly more inhibitive towards SKOV3 cells, which is a drug resistant ovarian cancer cells, than towards A2780 cells (a cell line is sensitive to cisplatin). After being incubated with adenocarcinoma (HeLa) and osteosarcoma cells (Saos-2), D-10 exhibited the IC₅₀ values of 27 μg/mL and 44 μg/mL, respectively (FIGS. 20F-G and 21B). In contrast, the IC₅₀ values of L-10 against HeLa and Saos-2 were 53 μg/mL and 80 μg/mL, respectively (FIGS. 20F-G and 21A). The cytotoxicity of L-10 and D-10 on drug sensitive (MES-SA) and drug resistant (MES-SA/D×5) uterine sarcoma cells was also tested. Both L-10 and D-10 exhibited high inhibitory activities on MES-SA cells with IC₅₀ values at 40 μg/mL and 31 μg/mL, respectively (FIGS. 20H and 21A-B). However, both the L-10 and D-10 showed lower cytotoxicity on MES-SA/D×5 cells, with the IC₅₀ values at 322 μg/mL and 163 μg/mL, respectively (FIGS. 20J and 21A-B). The incubation of L-10 on melanoma cancer cells (A375) and hepatocellular carcinoma cells (HepG2) resulted in the IC₅₀ values at 94 μg/mL and 97 μg/mL, respectively (FIGS. 20I, 20K, and 21A). D-10 showed higher cytotoxicity, inhibiting A375 and HepG2 cells with and IC₅₀ values of 38 μg/mL and 88 μg/mL, respectively (FIGS. 20I, 20K, and 21B). Being tested on two glioblastoma cell line cell lines (U87MG and T98G), L-10 showed little cytotoxicity toward these two cancer cell lines at or below concentration of 146 μg/mL (FIGS. 20L-M, and 21A). The cell viabilities of U87MG and T98G cells, after being treated by 365 μg/mL of L-10, were about 70% and 60%, respectively. D-10 exhibited higher inhibitory activity towards to these two cell lines, with the IC₅₀ values of 126 μg/mL and 145 μg/mL at 72 hours incubation of U87MG and T98G, respectively (FIGS. 20L-M, and 21B). As a control experiment, the cytotoxicity of the precursors was tested on two normal cell lines—HS-5, a stromal cell derived from normal bone marrow, and PC12, a model neuron cell line from mice. Both L-10 and D-10 showed little cytotoxicity on these two cell lines (FIGS. 20N-O and 21A-B). The IC₅₀ values of L-10 on HS-5 and PC-12 were above 73 μg/mL, which were higher than the IC₅₀ values of L-10 on most of the cancer cell lines tested. The IC₅₀ values of D-10 on HS-5 and PC-12 were 50 and 62 μg/mL, respectively, which were also two to three times higher than the IC₅₀ values of D-10 against most of the cancer cells tested.

To assess the selectivity of L-10 and D-10 towards cancer cells, their activities on drug resistant ovarian cancer cells (A2780cis and SKOV3) and the stromal cells (HS-5) at about the IC₉₀ of the inhibitors against the cancer cells were compared. As shown in FIG. 22, L-10, at 73 μg/mL, showed high cytotoxicity on both A2780cis and SKOV3 cells (ovarian cancer cells) and inhibited almost 100% of the cancer cells on the third day. At the same concentration, L-10 hardly inhibited the HS-5 cell (and the cell viability is about 80% on the third day). D-10 also showed potent cytotoxicity towards ovarian cancer cells, especially to SKOV3 cells. D-10, at 37 μg/mL, inhibited more than 90% of SKOV3 cells and about 55% of A2780cis cells on the third day. Treated by D-10 at 37 μg/mL, HS-5 cells exhibited about 80% viability at day three. These results confirmed that the two precursors are able to selectively inhibit ovarian cancer cells at their IC₉₀ without inhibiting HS-5 cells.

As another positive control, the cytotoxicity of cisplatin on those two ovarian cancer cell lines was tested. Cisplatin, which is known as a powerful anticancer drug, inhibited 90% of SKOV3 cells and about 98% of A2780cis cells on the third day at concentration of 37 μg/mL (FIG. 22). However, cisplatin (37 μg/mL) killed almost 100% of HS-5 on the third day (FIG. 22). This result indicated the limitation of cisplatin, confirming that cisplatin has little selectivity and may cause high systematic toxicity to normal cells. Interestingly, L-10, at 37 μg/mL, was innocuous to those two ovarian cancer cells, as well as to normal cells. This result indicated that the proteolytic instability of L-10 in cellular environment likely contributes to its low cytotoxicity.

To assess whether D-10 can enhance the efficacy of alternative cisplatin doses, the cytotoxicity of combinations of high and low D-10 doses (20 or 100 μM) with various lower cisplatin doses (10, 20 or 50 μM cisplatin) was assessed using the MTT assay. As shown in FIG. 23, 100 μM doses of D-10 enhanced the efficacy of 10, 20 or 50 μM cisplatin doses in comparison to those same doses of cisplatin alone. This was apparent on each of days 1 to 3.

To elucidate the modality of cell death (Vanden Berghe et al., “Molecular Crosstalk Between Apoptosis, Necroptosis, and Survival Signaling,” Molecular & Cellular Oncology 2(4):e975093 (2015), which is hereby incorporated by reference in its entirety), caused by intracellular enzyme-induced assembly, the pan caspase inhibitor Z-VAD-FMK (Susin et al., “Molecular Characterization of Mitochondrial Apoptosis-Inducing Factor,” Nature 397(6718):441-46 (1999), Slee et al., “Benzyloxycarbonyl-val-ala-asp (ome) Fluoromethylketone (z-vad-Fmk) Inhibits Apoptosis by Blocking the Processing of cpp32,” Biochem. J. 315:21-24 (1996), which are hereby incorporated by reference in their entirety) was used to treat the cancer cells together with L-10 and D-10 to test the roles of caspases in the cell death process. Z-VAD-FMK is an anti-apoptotic agent that can inhibit caspase activities. As shown in FIG. 24A-D, there was hardly obvious difference with and without the addition of Z-VAD-FMK (45 μM) into the L-10 treated SKOV3 or A2780cis cells. This result indicated that EIA of L-10 caused death of SKOV3 or A2780cis via caspase-independent mechanism (Broker et al., “Cell Death Independent of Caspases: A review,” Clin. Cancer Res. 11(9):3155-62 (2005), which is hereby incorporated by reference in its entirety). Contrasting to the case of L-10, the addition of Z-VAD-FMK (45 μM) significantly increased the viability of the SKOV3 cell treated the D-10 (37 μg/mL) from 10% to 77%. However, Z-VAD-FMK hardly increased the cell viability when 73 μg/mL of D-10 treated the SKOV3 cells. These results indicate that the mechanism of SKOV3 cell death depends on the amount of intracellular nanofibrils formed by enzyme-induced assembly. While D-10 at 37 μg/mL largely leads to apoptosis, D-10 at 73 μg/mL apparently caused caspase independent cell death of SKOV3. In the case of A2780cis cells treated by D-10 at 37 μg/mL, the addition of Z-VAD-FMK resulted in only insignificant increase (10%) cell viability, indicating that apoptosis unlikely is the major cause of the death of A2780cis cells.

To further verify that intracellular enzyme-induced assembly selectively inhibits cancer cells, the stromal cells (HS-5) cells were co-cultured together with the drug resistant ovarian cancer cells (SKOV3 or A2780cis cells). 5×10³ of SKOV3 cells (or A2780cis) and 5×10³ HS-5 cells per well were seeded together and co-cultured in DMEM medium. Based on the results in FIG. 22, the concentrations of L-10 and D-10 at 73 and 37 μg/mL, respectively, were chosen. As shown in FIG. 25A, precursor L-10 at 73 μg/mL showed cytotoxicity to the co-cultured HS-5 and SKOV3 cells, which causes about 60% of cell death on the third day. The efficacy of L-10 (73 μg/mL) on co-cultured HS-5 and A2780cis cells was comparable to its efficacy on co-cultured HS-5 and SKOV3 cells as it inhibited about 57% of cells on the third day. The inhibitory activities of L-10 and D-10 towards the co-culture agree well with their cytotoxicity against A2780cis, SKOV3, and H-5 cells. D-10 (37 μg/mL) was used to treat the co-cultured cells and it was found that the inhibitory activity of D-10 increased when the incubation time was extended to day three. For example, on the first day, the cell viabilities of co-cultured HS-5 and SKOV3 cells and co-cultured HS-5 and A2780cis cells treated by D-10 (37 μg/mL) were 79% and 80%, respectively. While on the third day, the cell viabilities dropped to 44% and 57%, respectively. In addition, D-10 (37 μg/mL) showed higher cytotoxicity to co-cultured HS-5 and SKOV3 cells than co-cultured HS-5 and A2780cis cells, which is consistent with the respective cytotoxicities of D-10 against only SKOV3 or only A2780cis cells. Contrary to the case of D-10, the efficacies of L-10 were almost the same between day two and day three (FIG. 25A), which agreed with the lower in vivo stability of L-10 than that of D-10.

To evaluate the contribution of the expression of CES for the observed selectivity against the cancer cells, the esterase activities in multiple cell lines were quantified (FIG. 25B). Using 6-CFDA (6-carboxyfluorescein diacetate) as the substrate of esterase, the fluorescence upon the hydrolysis by intracellular esterases was measured. For the comparison, the intensity of the measured fluorescence was divided by the total cellular proteins (pg/cell) of each cell line. HepG2 and A2780 showed relatively higher esterase activity among the tested cell lines, with values larger than 1. HCC1937, SKOV3 and HeLa cells showed similar esterase activities, which were higher than 0.8. A2780cis cells have an esterase activity value higher than 0.7 and U87MG, T98G, A375, MES-SA and MCF-7 cells have values around 0.6. MES-SA/D×5 cells have very low esterase activity value at about 0.4 and HS-5 cells have the lowest esterase activity (about 0.35) among all the cell lines tested. The trend of the esterase activity largely matched the cytotoxicity results shown in FIG. 20A-O and FIGS. 21A-B. For example, both the precursors showed low cytotoxicity to HS-5 cells and MES-SA/D×5 cells, which had low esterase activity values, whereas the precursors showed high cytotoxicity to A2780, HCC1937, SKOV3, HeLa and A2780cis cells, which have comparably higher esterase activity values. This confirms that CES plays an important role in selectively inhibiting cancer cells.

This Example confirms that cytosolic enzyme-instructed self-assembly is a viable anticancer treatment against a wide range of cancer types that overexpress endoenzymes, including esterases. This fundamentally new approach harnesses the enzymatic difference between cancer and normal cells for overcoming cancer drug resistance. Delivery of taurine or hypotaurine conjugated peptides capable of forming intramolecular nanofibers by enzyme-induced assembly, importantly, can be achieved without vehicles, and can cause cytotoxicity via apoptosis or necroptosis.

### Example 7—In Vivo Inhibition of Cancer Cell Tumor by D-10

As demonstrated in Example 5 (FIGS. 18A-C), D-10 is well tolerated at doses of 20 mg/kg, and even possibly higher. Therefore, D-10 will be used in an animal model of cancer.

A2780-cp cells are an established model cell-line for evaluating the efficacy and toxicity of new drugs in vivo. From preliminary studies, A2780-cp cells are known to form neoplasms in nude mice with moderate growth rate. 3 weeks after injection of 1.0×10⁷ cells, neoplasm can be ready intraperitoneal of the nude mice. After another 4 weeks of growth, average tumors will reach about 10 mm in diameter. The histopathologic study of these tumors indicates well-circumscribed neoplasms with moderate host cell infiltration. The tumors are typically well encapsulated by a fibrous capsule and are rarely invasive into the surrounding tissue or the muscle of the body wall. Tumor metastases are rarely seen.

Six nude mice will be used for pilot experiments to define the tumor growth curve, and establish appropriate endpoints of the experiments. 1.0×10⁷ A2780-cp cells will be implanted onto the mice. Three of the mice will be used to define the tumor growth curve without any treatment. Three of the mice will be given 100 μL of 100 nM Taxol every other day, starting from three weeks after the implantation of tumor cells. Endpoint will be set at the time, 4 or 5 weeks after the injection of drugs, or body condition score of <1, body condition score of <2 and decreases in activity, grooming, eating, drinking or nest building in noted, weight loss exceeding 15%, weight gain exceeding 5 g, anorexia, and/or diarrhea, whichever comes first. The mice will be sacrificed after the pilot experiment by inhalation of canister CO₂ via chamber followed by cervical dislocation.

22 six-week-old female nude mice will be used for testing of each compound. They will be divided into 4 groups: one group will be given normal saline solution as negative control; one group will be given commercial taxol as positive control; one test group will be given D-10 at 500 μM; and the other group will be given D-10 at 5 mM. For statistical significance, there must be no less than 3 nude mice in each of the three groups. As such, 5 mice will be used for each group. Considering that the success rate of cancer cell-line transplantation is not 100%, 2 more nude mice will be used as backup. If more than 12 mice developed tumors, the extra ones will be introduced into the test group.

Harvest Cells from Culture:

healthy A2780-cp cells (origin from human ovary) will be collected and diluted into concentration of 2.0×10⁷ cells/mL of medium. These cell suspensions will be sealed in a sterile tube and kept at 37° C. before use. This step will be performed in a BS2 hood.

Tumor implantation: The inoculation area of the mouse will be cleaned and disinfected with 70% v/v ethanol. The cell suspension (500 μL, 1.0×10⁷ cells per injection) will be withdrawn from the sterile tube into a 1-cc TB syringe (needle removed). The mouse is tilted with its head slightly toward the ground so that its head is lower than it hind end. This allows the abdominal viscera to shift cranially and minimize accidental puncture of abdominal organs at site of injection. The cell suspension will be injected intraperitoneally of the mouse with a 25 G needle. Pull back on the plunger will ensure negative pressure prior to injecting. If there is negative pressure, proceed with the injection by depressing the plunger until the solution has been fully administered. After the injection, the mice will be placed in a clean cage and under observation for 10 to 15 min to ensure that there is no untoward effect.

Compound treatment: The treatment will start after the tumor cells are implanted intraperitoneally for three weeks. The nude mice will be randomly separated into 4 groups. Hydrogelator precursor D-10 (500 μM and 5 mM) for test group, PBS for negative control group and Taxol (100 nM) for positive control group will be applied, respectively, by intraperitoneal injection with a fixed volume of 100 μL. After the injection, the mice will be placed in a clean cage and observe for 10 to 15 min to ensure there is no untoward effects. The injection will be performed every three days. The therapy will be ended after 4 weeks of treatment, or when endpoint is reached, whichever comes first.

Side effects: The maximum tolerated dose (MTD) of taxol in nude mice is 60 mg/kg. The dose to be given is 100 μg/kg which is much lower than the MTD. And based on preliminary experiments, such low doses do not introduce any visible side effects. The test compound, D-10, is peptide based and of low molecular weight. In vitro and in vivo toxicity tests in the preceding Examples proved that these compounds have very low toxicity on various cell lines and in animals. Thus, no side effects are expected for the test compounds on nude mice.

Measurements: Tumor size: After the end point is reached, the mice will be sacrificed. With necropsy of the mice, the ovarian tumors will be retrieved and their sizes will be measured. Body and tumor weight: The body weight of the mice will be measured and recorded along with the injection of drugs. Finally, after retrieving the ovarian tumors, the retrieved tumors will be weighed. The size and weight of some organs (e.g. spleen, or kidney) from the sacrificed mice will also be measured (see FIGS. 18A-C).

Endpoint: The total time between tumor implantation and euthanasia is estimated at 7 to 8 weeks. Survival time reflects this time required for the mice to reach any endpoints as noted above. At the end of studies mice will be euthanized.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims

1. A peptide comprising a plurality of amino acid residues and an enzymatically cleavable moiety comprising a taurine or hypotaurine residue, the enzymatically cleavable-moiety being linked to the peptide via covalent bond, wherein the peptide is capable of self-assembly to form nanofibrils in the presence of an enzyme that hydrolyzes the enzymatically cleavable-moiety.

2. The peptide according to claim 1, wherein the amino acids are aromatic amino acids selected from the group consisting of phenylalanine, phenylalanine derivatives, tyrosine, tyrosine derivatives, tryptophan, and tryptophan derivatives.

3. The peptide according to claim 1, wherein the amino acids are all D-amino acids or all L-amino acids.

4. The peptide according to claim 1, wherein the amino acids are a mixture of L-amino acids and D-amino acids.

5. The peptide according to claim 1, wherein a taurine residue is present.

6-7. (canceled)

8. The peptide according to claim 1, wherein the covalent bond linking the enzymatically cleavable-moiety to the peptide is a peptide bond.

9. The peptide according to claim 1, wherein the enzymatically cleavable-moiety further comprises an ester, a carbonate, a thiocarbonate, a carbamate, a carboxylate, a diacyl anhydride, or an amide bond.

10. The peptide according to claim 9, wherein the enzymatically cleavable-moiety comprising an ester bond is 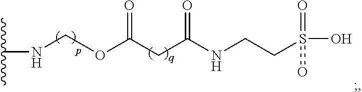
wherein the enzymatically cleavable-moiety comprising a carbonate bond is 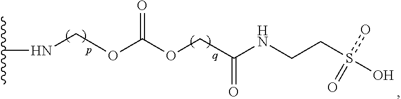
wherein the enzymatically cleavable-moiety comprising a thiocarbonate bond is 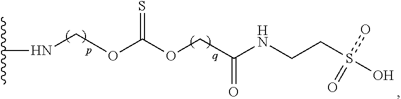
wherein the enzymatically cleavable-moiety comprising a carbamate bond is 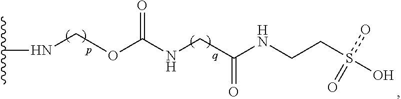
wherein the enzymatically cleavable-moiety comprising a carboxylate bond is 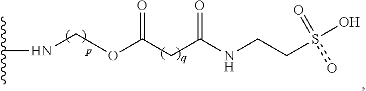
or
wherein the enzymatically cleavable-moiety comprising an amide bond is 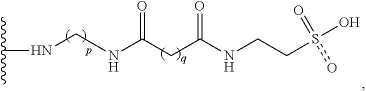
and
in each of the structures above p and q are independently integers from 1 to 5.

11-13. (canceled)

14. The peptide according to claim 1, wherein the N-terminal amino acid of the peptide is capped by a capping moiety.

15-16. (canceled)

17. The peptide according to claim 14, wherein the capping moiety comprises a fluorophore; an aromatic group; a cytotoxic agent selected from the group of chemotherapeutic agents, antiangiogenic agents, and immunomodulating agents; an antigen; an alkylacyl group; an arylacyl group; or an acylated nucleoside.

18. The peptide according to claim 1, wherein one of the amino acids comprises a sidechain conjugated to a fluorophore; a cytotoxic agent selected from the group of chemotherapeutic agents, antiangiogenic agents, and immunomodulating agents; or an antigen.

19-23. (canceled)

24. The peptide according to claim 1, wherein said peptide is between 2 to 10 amino acids.

25. The peptide according to claim 1, wherein the peptide is selected from the group consisting of: 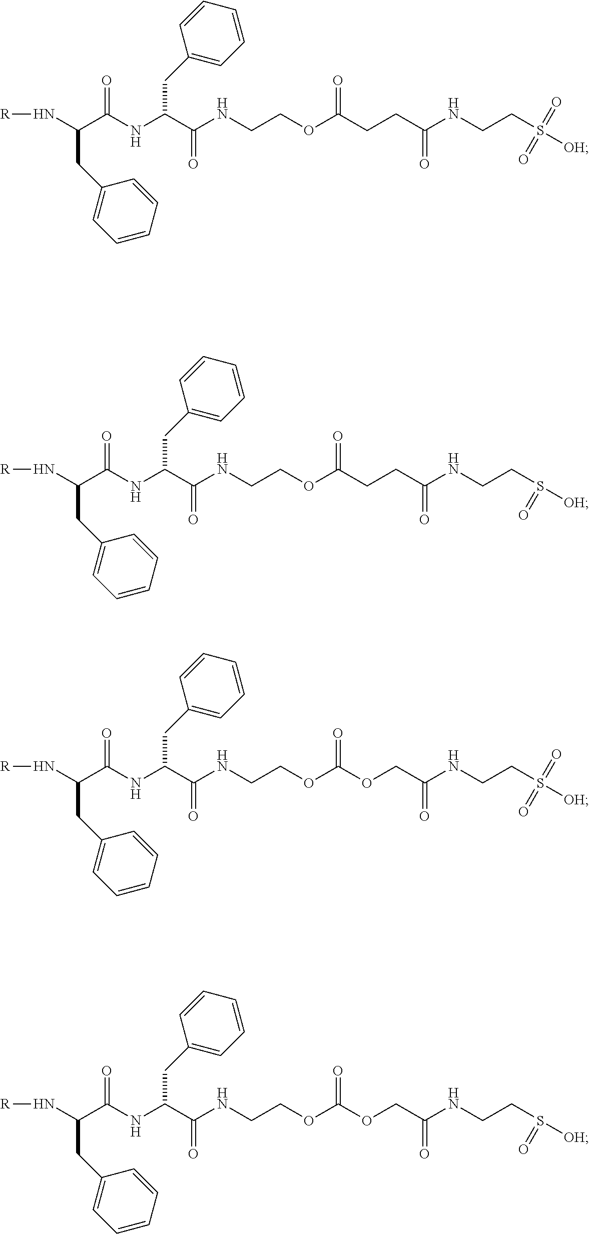 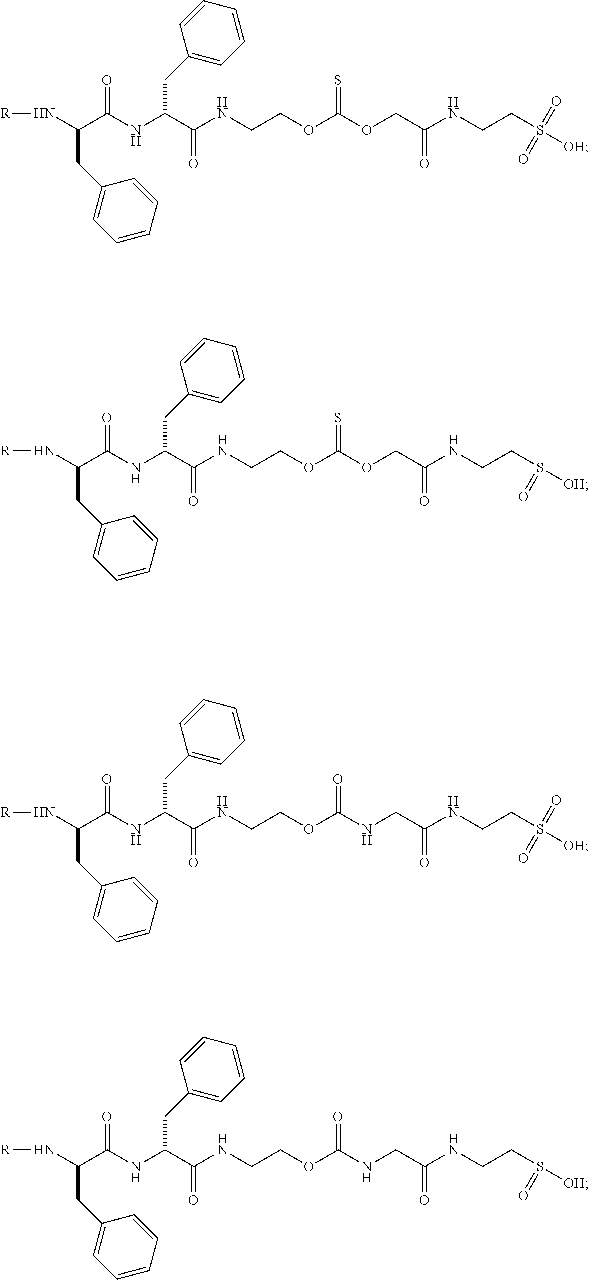 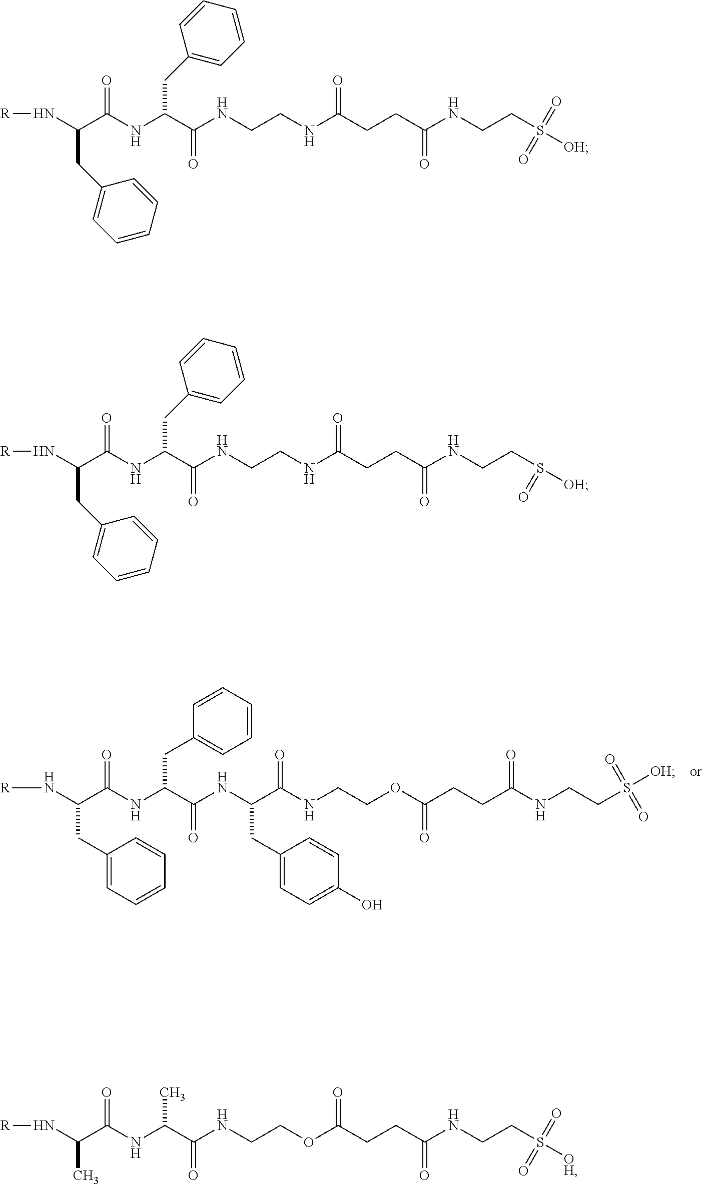
wherein in each of the above structures R can be H, an alkylacyl, an arylacyl, a fluorophore, a cytotoxic drug, a nucleobase, or a nucleoside analog, optionally linked to the N-terminal group of the peptide via a linker.

26. (canceled)

27. A pharmaceutical composition comprising a pharmaceutically acceptable carrier and a peptide according to claim 1.

28. (canceled)

29. The pharmaceutical composition according to claim 27 wherein the carrier is an aqueous medium.

30. The pharmaceutical composition according to claim 27, wherein the peptide is present at a concentration of about 1 μM to about 10 mM.

31. (canceled)

32. A method for treating a cancerous condition comprising:
administering to a subject having a cancerous condition a therapeutically effective amount of the peptide according claim 1, wherein said administering is effective to cause uptake of the peptide by the cancer cells and intracellular self-assembly of the peptides to form a nanofibril network upon enzymatic cleavage of the enzymatically cleavable-moiety.

33-34. (canceled)

35. The method according to claim 32, wherein said administering is carried out parenterally, subcutaneously, intravenously, intradermally, intramuscularly, intraperitoneally, by implantation, by intracavitary or intravesical instillation, intraarterially, intralesionally, intradermally, peritumorally, intratumorally, or by introduction into one or more lymph nodes.

36-37. (canceled)

38. The method according to claim 32, wherein the peptide is conjugated with a chemotherapeutic agent, an antiangiogenic agent, or an immunomodulating agent.

39. The method according to claim 32 further comprising:
administering to the subject a chemotherapeutic agent, an immunotherapeutic agent, or a radiotherapeutic agent.

40. The method according to claim 39 wherein the chemotherapeutic agent is a platinum-containing chemotherapeutic.

41-44. (canceled)

45. The method according to claim 32, wherein the subject is a human.

46. The method according to claim 32, wherein the cancer cell is present in a solid tumor.

47. (canceled)

48. The method according to claim 32, wherein the cancerous condition is selected from the group of cancers or neoplastic disorders of the brain and CNS, pituitary gland, breast, blood, lymph node, lung, skin, bone, head and neck, oral, eye, gynecological tissues, genitourinary, and gastrointestinal.

49. The method according to claim 32, wherein the peptide or pharmaceutical composition is administered with a peptide dose of between about 1 μg to about 100 mg.

50. A method for forming a nanofibril network internally of cancer cells, the method comprising:
contacting a cancer cell that expresses an endoenzyme having esterase/hydrolase activity with the peptide according to claim 1, wherein said contacting is effective to cause self-assembly of the peptides to form an intracellular nanofibril network within the cancer cell.

51-64. (canceled)

65. A method of making a peptide comprising:
providing a peptide comprising a plurality of amino acid residues covalently linked to an enzymatically cleavable moiety having a terminal reactive group, and
reacting the peptide with taurine or hypotaurine to form the peptide according to claim 1.

66. The method according to claim 65, wherein the terminal reactive group is a carboxylic acid and the taurine or hypotaurine forms an amide bond with the enzymatically cleavable moiety of the peptide.

67-69. (canceled)
